SEQUENCE LISTING (4 sequences)

SEQ ID NO: 1 - RNA, 22 nt, Homo sapiens
cgucuuacccagcaguguuugg

SEQ ID NO: 2 - DNA, 22 bp, Artificial sequence
  Synthetic sequence  Capture 200c-5p
ccaaacactgctgggtaagacg

SEQ ID NO: 3 - DNA, 23 bp, Artificial sequence
  Synthetic sequence Capture 17-5p
ctacctgcactgtaagcactttg

SEQ ID NO: 4 - DNA, 22 bp, Artificial
  Synthetic sequence Capture let7a-5p
aactatacaacctactacctca

(19) United States
(12) Patent Application Publication
(10) Pub. No.: US 2023/0194472 A1
(43) Pub. Date: Jun. 22, 2023

(54) MICRORNA ANALYSIS USING TUNNELING CURRENT

(71) Applicant: Osaka University, Suita-shi, Osaka (JP)

(72) Inventors: Masateru Taniguchi, Suita-shi, Osaka (JP); Hideshi Ishii, Suita-shi, Osaka (JP); Takahito Ohshiro, Suita-shi, Osaka (JP); Masamitsu Konno, Suita-shi, Osaka (JP)

(21) Appl. No.: 17/605,917

(22) PCT Filed: Apr. 24, 2020
(86) PCT No.: PCT/JP2020/017654

§ 371 Date: Oct. 22, 2021

(30) Foreign Application Priority Data
Apr. 26, 2019 (JP) 2019-085805

(51) Int. Cl. G01N 27/62; C12Q 1/6886

(52) U.S. Cl. CPC G01N 27/62; C12Q 1/6886

(57) ABSTRACT

The present disclosure provides a method for analyzing a microRNA using a tunneling current. The present disclosure provides a method for identifying the base sequence and/or modification state of a microRNA using a tunneling current, and a system and a program to be used in the method. Furthermore, the present disclosure provides a method for analyzing the conditions of a subject, said method comprising determining the base sequence and/or modification state of a microRNA using a tunneling current. For example, methylation modification can be analyzed thereby.

### TECHNICAL FIELD

The present disclosure relates to a method of identifying a base sequence and/or modification state of a microRNA by using a tunneling current and an application thereof. The present disclosure also relates to identifying a base sequence and/or modification state of a microRNA by using a tunneling current and a system and program for use in an application thereof. Furthermore, the present disclosure relates to a method of analyzing a condition of a subject comprising identifying a base sequence and/or modification state of a microRNA by using a tunneling current and an application thereof.

### BACKGROUND ART

The technology of analyzing a base sequence of a polynucleotide is applied in the fields of not only academic research, but also medicine, drug discovery, crime investigation, and the like. Interest in the development of such a technology has been increasing.

Conventional polynucleotide (specifically DNA) sequencers are based on an optical measurement technology, which identifies a fluorescent label. Such sequencers do not directly identify constituent nucleotides of a polynucleotide themselves. Thus, analysis of the base sequence of a polynucleotide with a conventional sequencer requires PCR using said polynucleotide as a template and addition of a fluorescent label to the polynucleotide extended by said PCR. This procedure not only requires a large number of reagents, but also is time consuming. Therefore, analysis of the base sequence of a polynucleotide with a conventional sequencer is very capital and time intensive.

In this regard, attempts have been made in the past dozen years to develop a technology for directly analyzing nucleotides constituting a polynucleotide with a single molecule of polynucleotide. For instance, an attempt has been made to develop a technology for analyzing the base sequence of a polynucleotide by detecting an ion current using a nanoscale pore (hereinafter, referred to as “nanopore”) of a chemically designed a hemolysin (see Non Patent Literature 1). However, such a technology has many problems, such as (1) limited selection of pore sizes and (2) instability of the system, so that prospect of use in practical application is not in sight.

It is understood that analysis of microRNAs with a short base length is particularly difficult.

### CITATION LIST

### Non Patent Literature

[NPL 1] J. Li, D. Stein, C. McMullan, D. Branton, M. J. Aziz, J. A. Golovchenko, Nature 412, 166 (2001)

### SUMMARY OF INVENTION

### Solution to Problem

The inventors completed the present invention by finding that the base sequence and/or modification state (e.g., presence/absence of a modification, type of modification, position of modification, etc.) of a microRNA can be identified by using a tunneling current. The present disclosure provides a method of identifying a base sequence and/or modification state of a microRNA by using a tunneling current and a system and program for use in the method. The present disclosure also provides a method of analyzing a condition of a subject comprising identifying a base sequence and/or modification state of a microRNA by using a tunneling current.

Therefore, the present disclosure provides the following.

### (Item 1)

A method of analyzing a modification state of a microRNA, comprising:

(A) passing the microRNA between an electrode pair;
(B) detecting a tunneling current that is generated when the microRNA passes between the electrode pair; and
(C) analyzing the modification state based on a pulse.

### (Item 2)

The method of any of the preceding items, wherein a modified position on a base sequence of the microRNA is identified.

### (Item 3)

The method of any of the preceding items, wherein a modified position on a chemical structure of the microRNA is identified.

### (Item 4)

The method of any of the preceding items, wherein a modification ratio of the microRNA is identified.

### (Item 5)

The method of any of the preceding items, wherein a modification ratio at a specific modified position of the microRNA is identified.

(Item 6) The method of any of the preceding items, wherein the modification comprises methylation.

### (Item 7)

The method of any of the preceding items, wherein a base sequence of the microRNA is at least partially identified.

### (Item 8)

The method of any of the preceding items, wherein both a base sequence and a modification state of the microRNA are analyzed.

### (Item 9)

The method of any of the preceding items, comprising referring to a result of analysis of the microRNA by a mass spectrometer to associate the modification state with the pattern of the tunneling current.

### (Item 10)

The method of any of the preceding items, comprising accumulating data on a combination of the modification state and the pattern of the tunneling current that have been associated.

### (Item 11)

The method of any of the preceding items, wherein the microRNA is present in a sample.

### (Item 12)

The method of any of the preceding items, comprising associating the condition of the subject from whom the microRNA has been obtained with the modification state.

### (Item 13)

A database constructed with data accumulated by the method of any of the preceding items.

### (Item 14)

A method of analyzing a subject, the method comprising:

(X) preparing a sample from a subject so that the sample comprises a microRNA derived from the subject;
(Y-A) passing the sample between an electrode pair;
(Y-B) detecting a tunneling current that is generated when the sample passes between the electrode pair;
(Y-C) analyzing the modification state based on the tunneling current; and
(Z) analyzing a condition of the subject based on the modification state.

### (Item 15)

The method of any of the preceding items, wherein the condition of the subject from whom the microRNA has been obtained is analyzed by referring to accumulated data on a combination of the modification state and a pattern of the tunneling current to analyze the modification state of the microRNA based on the detected pattern of the tunneling current.

### (Item 16)

The method of any of the preceding items, wherein a result of analyzing the condition of the subject from whom the sample has been obtained is shown in 15 minutes or less from the time the sample was subjected to tunneling current measurement.

### (Item 17)

The method of any of the preceding items, wherein a condition of cancer, an inflammatory bowel disease, Crohn's disease, diabetes, or a psychiatric disease of a subject is analyzed.

### (Item 18)

A program configured to implement, on a computer, a method of analyzing a microRNA, comprising: inputting a result of analysis of a microRNA by a mass spectrometer; inputting a pattern of a tunneling current obtained by tunneling current measurement on the microRNA; and determining a modification state of the microRNA by associating the result of analysis by mass spectrometry with the pattern of the tunneling current.

### (Item 19)

A program configured to implement, on a computer, a method of analyzing a microRNA, comprising: referring to accumulated data on a combination of a modification state of a microRNA and a pattern of a tunneling current obtained by tunneling current measurement on the microRNA to show a modification state of the microRNA of a subject from whom the microRNA has been obtained based on the pattern of the tunneling current obtained by tunneling current measurement on the microRNA.

### (Item 20)

A program configured to implement, on a computer, a method of analyzing a subject, the method comprising: obtaining a pattern of a tunneling current by tunneling current measurement on a microRNA of the subject; referring to a database comprising a combination of a modification state of the microRNA and a pattern of a tunneling current already obtained by tunneling current measurement to analyze the modification state of the microRNA of the subject based on the obtained pattern of the tunneling current; and analyzing a condition of the subject based on the modification state.

### (Item 21)

A system for associating a modification state of a microRNA with a pattern of a tunneling current obtained by tunneling current measurement, comprising:

a mass spectrometer;

a tunneling current meter; and

an analysis/determination unit for analyzing and determining a modification state of a microRNA of interest by associating results of measuring the microRNA of interest by the mass spectrometer and the tunneling current measurement.

### (Item 22)

A system for determining a condition of a subject based on a modification state of a microRNA, comprising:

a tunneling current meter; and

a modification analysis/determination unit for referring to accumulated data on a combination of a modification state of a microRNA and a pattern of a tunneling current obtained by tunneling current measurement to analyze and determine a modification state of a microRNA of a subject from whom the microRNA has been obtained based on the pattern of the tunneling current obtained by tunneling current measurement on the microRNA.

### (Item 23)

The system of any of the preceding items, comprising a condition analysis/determination unit for analyzing and determining the condition of the subject based on the analyzed and determined modification state.

### (Item A1)

A method of analyzing a modification state of a microRNA, comprising:

(A) passing the microRNA between an electrode pair;
(B) detecting a tunneling current that is generated when the microRNA passes between the electrode pair; and
(C) analyzing the modification state based on a pulse.

### (Item A2)

The method of any of the preceding items, wherein a modified position on a base sequence of the microRNA is identified.

### (Item A3)

The method of any of the preceding items, wherein a modified position on a chemical structure of the microRNA is identified.

### (Item A4)

The method of any of the preceding items, wherein the modification comprises methylation.

### (Item A5)

The method of any of the preceding items, wherein a base sequence of the microRNA is at least partially identified.

### (Item A6)

The method of any of the preceding items, wherein both a base sequence and a modification state of the microRNA are analyzed.

### (Item A7)

The method of any of the preceding items, comprising referring to a result of analysis of the microRNA by a mass spectrometer to associate the modification state with the pattern of the tunneling current.

### (Item A8)

The method of any of the preceding items, comprising accumulating data on a combination of the modification state and the pattern of the tunneling current that have been associated.

### (Item A9)

The method of any of the preceding items, wherein the microRNA is present in a sample.

### (Item A10)

The method of any of the preceding items, comprising associating a condition of a subject from whom the microRNA has been obtained with the modification state.

### (Item A11)

A database constructed with data accumulated by the method of any of the preceding items.

### (Item A12)

A method of analyzing a subject, the method comprising:

(X) preparing a sample from the subject so that the sample comprises a microRNA derived from the subject;
(Y-A) passing the sample between an electrode pair;
(Y-B) detecting a tunneling current that is generated when the sample passes between the electrode pair;
(Y-C) analyzing the modification state based on the tunneling current; and
(Z) analyzing a condition of the subject based on the modification state.

### (Item A13)

The method of any one of the preceding items, wherein the condition of the subject from whom the microRNA has been obtained is analyzed by referring to accumulated data on a combination of the modification state and a pattern of the tunneling current to analyze a modification state of the microRNA based on the detected pattern of the tunneling current.

### (Item A14)

The method of any of the preceding items, wherein a result of analyzing the condition of the subject from whom the sample has been obtained is shown in 15 minutes or less from the time the sample was subjected to tunneling current measurement.

### (Item A15)

The method of any of the preceding items, wherein a condition of cancer, an inflammatory bowel disease, Crohn's disease, diabetes, or a psychiatric disease of a subject is analyzed.

### (Item A16)

A program configured to implement, on a computer, a method of analyzing a microRNA, comprising: inputting a result of analysis of a microRNA by a mass spectrometer; inputting a pattern of a tunneling current obtained by tunneling current measurement on the microRNA; and determining a modification state of the microRNA by associating the result of analysis by mass spectrometry with the pattern of the tunneling current.

### (Item A17)

A program configured to implement, on a computer, a method of analyzing a microRNA, comprising: referring to accumulated data on a combination of a modification state of a microRNA and a pattern of a tunneling current obtained by tunneling current measurement on the microRNA to show the modification state of the microRNA of a subject from whom the microRNA has been obtained based on the pattern of a tunneling current obtained by tunneling current measurement on the microRNA.

### (Item A18)

A program configured to implement, on a computer, a method of analyzing a subject, the method comprising: obtaining a pattern of a tunneling current by tunneling current measurement on a microRNA of the subject; referring to a database comprising a combination of a modification state of the microRNA and the pattern of a tunneling current already obtained by tunneling current measurement to analyze the modification state of the microRNA of the subject based on the obtained pattern of the tunneling current; and analyzing a condition of the subject based on the modification state.

### (Item A19)

A system for associating a modification state of a microRNA with a pattern of a tunneling current obtained by tunneling current measurement, comprising:

a mass spectrometer;

a tunneling current meter; and

an analysis/determination unit for analyzing and determining a modification state of a microRNA of interest by associating results of measuring the microRNA of interest by the mass spectrometer and the tunneling current measurement.

### (Item A20)

A system for determining a condition of a subject based on a modification state of a microRNA, comprising:

a tunneling current meter; and

a modification analysis/determination unit for referring to accumulated data on a combination of a modification state of a microRNA and a pattern of a tunneling current obtained by tunneling current measurement to analyze and determine a modification state of a microRNA of a subject from whom the microRNA has been obtained based on the pattern of a tunneling current obtained by tunneling current measurement on the microRNA.

### (Item A21)

The system of any of the preceding items, comprising a condition analysis/determination unit for analyzing and determining the condition of the subject based on the analyzed and determined modification state.

The present disclosure is intended so that one or more of the features described above can be provided not only as the explicitly disclosed combinations, but also as other combinations thereof. Additional embodiments and advantages of the present disclosure are recognized by those skilled in the art by reading and understanding the following detailed description as needed.

### Advantageous Effects of Invention

According to the present disclosure, the base sequence and/or modification state (e.g., presence/absence of a modification, type of modification, position of modification, modification ratio, etc.) of a microRNA can be identified in a simple, quick, and/or accurate manner. The present disclosure can also provide a new method of determining a condition of a subject based on the base sequence and/or modification state of a microRNA.

### BRIEF DESCRIPTION OF DRAWINGS

FIG. 1 shows that a result of measuring a microRNA by tunneling current sequencing differs between an unmodified form and a modified form. The diagram on the top row of (A) shows the result of measuring unmodified synthetic 200c-5p, and the diagram on the bottom row of (A) shows the result of measuring modified synthetic 200c-5p (7^{th} base is methylated adenine, and 13^{th} base is methylated cytosine). The vertical axis in each of the diagrams of (A) represents relative conductance. The diagrams of (B) show a comparison of an unmodified form and a modified form by extracting reading results corresponding to the 7^{th} base (left) and the 13^{th} base (right) from the diagrams of (A), respectively.

FIG. 2 shows an example of identifying the presence/absence of a modification in a microRNA obtained from a sample by tunneling current sequencing. The figure shows, from the top in order, a result of measuring unmodified synthetic 200c-5p, a resulting of measuring 200c-5p obtained from a sample, and a result of measuring modified synthetic 200c-5p (7^{th} base is methylated adenine and 13^{th} base is methylated cytosine).

FIG. 3 shows analysis of the results of measuring samples in FIG. 2. In the diagram of (A), the vertical axis represents relative conductance. The diagrams of (B) are histograms showing the results of reading out the 7^{th} base (top) and the 13^{th} base (bottom), respectively. In the diagrams of (B), the vertical axis represents frequency, and the horizontal axis represents the relative conductance.

FIG. 4 shows examples of results of measuring a tunneling current of a nucleic acid. In the diagrams of (A), (B), and (C), the vertical axis indicates the detected tunneling current (pA), and the horizontal axis indicates the time (seconds). The diagram of (B) is an expanded view of the portion framed with a box in the diagram of (A). In the diagram of (C), the horizontal lines traversing the diagram indicate, from the bottom, the baseline value of tunneling current, the mode of the maximum current values for uracil, the mode of maximum current values for adenine, and the mode of the maximum current values for guanine. It can be seen that the base sequence of “UGAG” is measured at a portion of a pulse indicating a duration of about 15 milliseconds in the middle.

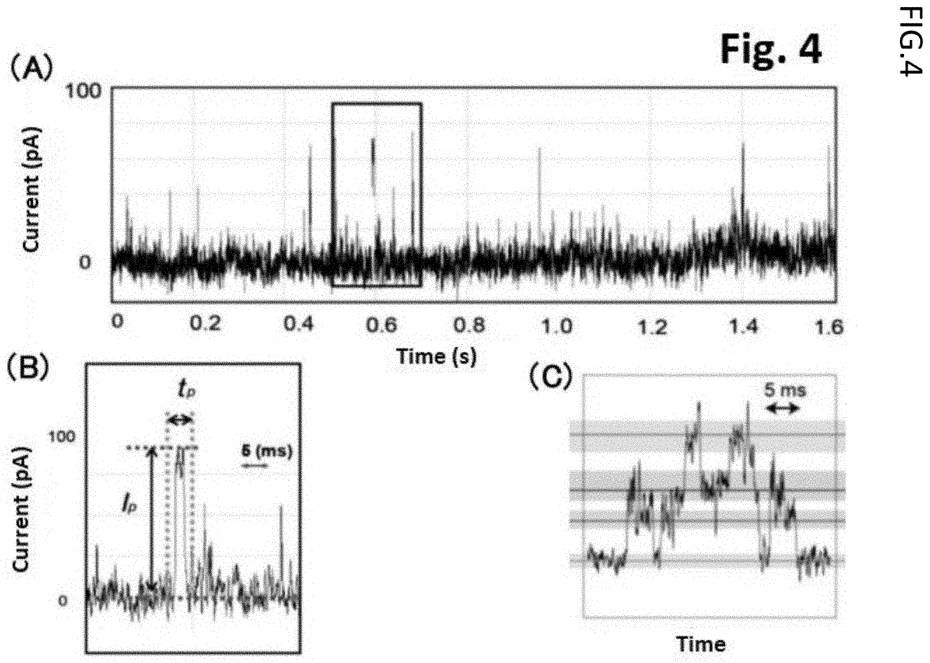

FIG. 5 is a schematic diagram of the configuration of a system.

FIG. 6 shows results of detecting the difference in modification states of microRNAs between a cancer patient and a healthy individual by tunneling current measurement. The top panel is the result of measuring concentrated let7a-5p, and the bottom panel is the result of measuring concentrated miR17-5p. Each panel shows a comparison of results for a pancreatic cancer patient (top) and healthy individual (bottom). The vertical axis represents relative conductance. The diagram shows whether there is a difference in the methylation ratios between a pancreatic cancer patient and a healthy individual for each adenine.

FIG. 7 shows results of tunneling current measurement on the top row and results of Hiseq measurement on the bottom row. The left column shows a comparison between a wild-type strain (DLD1) and an FTD resistant strain, and the right column shows a comparison between a wild-type strain and a 5-FU resistant strain. The vertical axis indicates the number of molecules counted in an experiment, which is the quantitative index of a microRNA.

FIG. 8 is a graph comparing 6 types of microRNAs in the comparison of 2 types of drug resistance in FIG. 6 (i.e., 12 plots each for wild-type strain (WT) and resistant strain (PS)) between tunneling current measurement and Hiseq measurement. The vertical axis indicates results of tunneling current measurement, and the horizontal axis indicates results of Hiseq measurement.

### DESCRIPTION OF EMBODIMENTS

The present disclosure is described hereinafter while showing the best mode of the present disclosure. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. The terms used herein should also be understood as being used in the meaning that is commonly used in the art, unless specifically noted otherwise. Thus, unless defined otherwise, all terminologies and scientific technical terms that are used herein have the same meaning as the general understanding of those skilled in the art to which the present disclosure pertains. In case of a contradiction, the present specification (including the definitions) takes precedence.

The definitions of the terms and/or basic technical matters especially used herein are described hereinafter when appropriate.

(Definitions, etc.)

As used herein, “ribonucleic acid (RNA)” refers to a molecule comprising at least one ribonucleotide residue. “Ribonucleotide” refers to a nucleotide with a hydroxyl group at position 2′ on β-D-ribofuranose moiety. Examples of RNA include mRNA, tRNA, rRNA, lncRNA, and miRNA.

As used herein, “microRNA (miRNA)” refers to a functional nucleic acid, which is encoded on the genome and ultimately becomes a very small RNA with a base length of 20 to 25 after undergoing a multi-stage production process. Specific information (sequence and the like) of miRNAs is available from, for example, mirbase (http://mirbase.org). For example, mature microRNAs in humans include those in the following table.

As used herein, “modification” used in the context of a nucleic acid refers to a substitution of a constituent unit of a nucleic acid or a part or all of the terminus thereof with another group of atoms, or addition of a functional group.

Examples of RNA modifications include, but are not limited to, those listed in the following tables. It is understood that anything can be used, as long as it falls under a modification.

TABLE 1

| Name | Abbreviation |
|---|---|
| 1,2′-O-dimethyladenosine | m1Am |
| 1,2′-O-dimethylguanosine | m1Gm |
| 1,2′-O-dimethylinosine | m1Im |
| 1-methyl-3-(3-amino-3-carboxypropyl)pseudouridine | m1acp3Y |
| 1-methyladenosine | m1A |
| 1-methylguanosine | m1G |
| 1-methylinosine | m1I |
| 1-methylpseudouridine | m1Y |
| 2,8-dimethyladenosine | m2,8A |
| 2-gelanylthiouridine | ges2U |
| 2-lysidine | k2C |
| 2-methyladenosine | m2A |
| 2-methylthio cyclic N6-threonylcarbamoyladenosine | ms2ct6A |
| 2-methylthio- N6-(cis-hydroxyisopentenyl)adenosine | ms2io6A |
| 2-methylthio- N6-hydroxynorvalylcarbamoyladenosine | ms2hn6A |
| 2-methylthio- N6-isopentenyladenosine | ms2i6A |
| 2-methylthio- N6-methyladenosine | ms2m6A |
| 2-methylthio- N6-threonylcarbamoyladenosine | ms2t6A |
| 2-selenouridine | se2U |
| 2-thio-2′-O-methyluridine | s2Um |
| 2-thiocytidine | s2C |
| 2-thiouridine | s2U |
| 2′-O-methyladenosine | Am |
| 2′-O-methylcytidine | Cm |
| 2′-O-methylguanosine | Gm |
| 2′-O-methylinosine | Im |
| 2′-O-methylpseudouridine | Ym |
| 2′-O-methyluridine | Um |
| 2′-O-methyluridine 5-oxyacetic acid methyl ester | mcmo5Um |
| 2′-O-ribosyladenosine (phosphoric acid) | Ar(p) |
| 2′-O-ribosylguanosine (phosphoric acid) | Gr(p) |
| 2′3′-cyclic phosphoric acid end | (pN)2′3′ > p |
| 3,2′-O-dimethyluridine | m3Um |

TABLE 1-2

| | |
|---|---|
| 3-(3-amino-3-carboxypropyl)-5,6-dihydrouridine | acp3D |
| 3-(3-amino-3-carboxypropyl)pseudouridine | acp3Y |
| 3-(3-amino-3-carboxypropyl)uridine | acp3U |
| 3-methylcytidine | m3C |
| 3-methylpseudouridine | m3Y |
| 3-methyluridine | m3U |
| 4-dimethylwyosine | imG-14 |
| 4-thiouridine | s4U |
| 5,2′-O-dimethylcytidine | m5Cm |
| 5,2′-O-dimethyluridine | m5Um |
| 5-(carboxyhydroxymethyl)-2′-O-methyluridine methyl | mchm5Um |
| ester | |
| 5-(carboxyhydroxymethyl)uridine methyl ester | mchm5U |
| 5-(isopentenylaminomethyl)-2-thiouridine | inm5s2U |
| 5-(isopentenylaminomethyl)-2′-O-methyluridine | inm5Um |
| 5-(isopentenylaminomethyl)uridine | inm5U |
| 5-aminomethyl-2-gelanylthiouridine | nm5ges2U |
| 5-aminomethyl-2-selenouridine | nm5se2U |
| 5-aminomethyl-2-thiouridine | nm5s2U |
| 5-aminomethyluridine | nm5U |
| 5-carbamoylhydroxymethyluridine | nchm5U |
| 5-carbamoylmethyl-2-thiouridine | ncm5s2U |
| 5-carbamoylmethyl-2′-O-methyluridine | ncm5Um |
| 5-carbamoylmethyluridine | ncm5U |
| 5-carboxyhydroxymethyluridine | chm5U |
| 5-carboxymethyl-2-thiouridine | cm5s2U |
| 5-carboxymethylaminomethyl-2-gelanylthiouridine | cmnm5ges2U |
| 5-carboxymethylaminomethyl-2-selenouridine | cmnm5se2U |
| 5-carboxymethylaminomethyl-2-thiouridine | cmnm5s2U |
| 5-carboxymethylaminomethyl-2′-O-methyluridine | cmnm5Um |
| 5-carboxymethylaminomethyluridine | cmnm5U |
| 5-carboxymethyluridine | cm5U |
| 5-cyanomethyluridine | cnm5U |
| 5-formyl-2′-O-methylcytidine | f5Cm |
| 5-formylcytidine | f5C |
| 5-hydroxycytidine | ho5C |

TABLE 1-3

| | |
|---|---|
| 5-hydroxymethylcytidine | hm5C |
| 5-hydroxyuridine | ho5U |
| 5-methoxycarbonylmethyl-2-thiouridine | mcm5s2U |
| 5-methoxycarbonylmethyl-2′-O-methyluridine | mcm5Um |
| 5-methoxycarbonylmethyluridine | mcm5U |
| 5-methoxyuridine | mo5U |
| 5-methyl-2-thiouridine | m5s2U |
| 5-methyaminomethyl-2-gelanylthiouridine | mnm5ges2U |
| 5-methylaminomethyl-2-selenouridine | mnm5se2U |
| 5-methylaminomethyl-2-thiouridine | mnm5s2U |
| 5-methylaminomethyluridine | mnm5U |
| 5-methylcytidine | m5C |
| 5-methyldihydrouridine | m5D |
| 5-methyluridine | m5U |
| 5-taurinomethyl-2-thiouridine | tm5s2U |
| 5-taurinomethyluridine | tm5U |
| 5′(3′-dephospho-CoA) | CoA(pN) |
| 5′(3′-dephosphoacetyl-CoA) | acCoA(pN) |
| 5′(3′-dephosphomalonyl-CoA) | malonyl-CoA(pN) |
| 5′(3′-dephosphosuccinyl-CoA) | succinyl-CoA(pN) |
| 5′ diphosphate end | p(pN) |
| 5′hydroxyl end | 5′-OH—N |
| 5′ monophosphate end | (pN) |
| 5′ nicotinamide adenine dinucleotide | NAD(pN) |
| 5′ triphosphate end | pp(pN) |
| 7-aminocarboxypropyl-dimethylwyosine | yW-86 |
| 7-aminocarboxypropylwyosine | yW-72 |
| 7-aminoacarboxypropylwyosine methyl ester | yW-58 |
| 7-aminomethyl-7-deazaguanosine | preQ1tRNA |
| 7-cyano-7-deazaguanosine | preQ0tRNA |
| 7-methylguanosine | m7G |
| 7-methylguanosine cap (cap 0) | m7Gpp(pN) |
| 8-methyladenosine | m8A |
| N2,2′-O-dimethylguanosine | m2Gm |
| N2,7,2′-O-trimethylguanosine | m2,7Gm |

TABLE 1-4

| | |
|---|---|
| N2,7-dimethylguanosine | m2,7G |
| N2,7-dimethylguanosine cap (cap DMG) | m2,7Gpp(pN) |
| N2,N2,2′-O-trimethylguanosine | m2,2Gm |
| N2,N2,7-trimethylguanosine | m2,2,7G |
| N2,N2,7-trimethylguanosine cap (cap TMG) | m2,2,7Gpp(pN) |
| N2,N2-dimethylguanosine | m2,2G |
| N2-methylguanosine | m2G |
| N4,2′-O-dimethylcytidine | m4Cm |
| N4,N4,2′-O-trimethylcytidine | m4,4Cm |
| N4,N4-dimethylcytidine | m4,4C |
| N4-acetyl-2′-O-methylcytidine | ac4Cm |
| N4-acetylcytidine | ac4C |
| N4-methylcytidine | m4C |
| N6,2′-O-dimethyladenosine | m6Am |
| N6,N6-2′-O-trimethyladenosine | m6,6Am |
| N6,N6-dimethyladenosine | m6,6A |
| N6-(cis-hydroxyisopentenyl)adenosine | io6A |
| N6-acetyladenosine | ac6A |
| N6-formyladenosine | f6A |
| N6-glycinylcarbamoyladenosine | g6A |
| N6-hydroxymethyladenosine | hm6A |
| N6-hydroxynorvalylcarbamoyladenosine | hn6A |
| N6-isopentenyladenosine | i6A |
| N6-methyl-N6-threonylcarbamoyladenosine | m6t6A |
| N6-methyladenosine | m6A |
| N6-threonylcarbamoyladenosine | t6A |
| Q base | Qbase |
| Adenosine | A |
| Agmatidine | C+ |
| α -dimethylmonophosphate cap | mm(pN) |
| α-methylmonophosphate cap | m(pN) |
| Archaeosine | G+ |
| Cyclic N6-threnonylcarbamoyladenosine | ct6A |
| Cytidine | C |
| Dihydrouridine | D |

TABLE 1-5

| | |
|---|---|
| Epoxyqueuosine | oQtRNA |
| Galactosyl-queuosine | galQtRNA |
| γ-methyltriphosphate cap | mpp(pN) |
| Glutamyl-queuosine | gluQtRNA |
| Guanosine | G |
| Guanosine added to any nucleotide | pG(pN) |
| Guanylated 5′ end (cap G) | Gpp(pN) |
| Hydroxy-N6-threonylcarbamoyladenosine | ht6A |
| Hydroxywybutosine | OHyW |
| Inosine | I |
| Isowyosine | imG2 |
| Mannosyl-queuosine | manQtRNA |
| Methylated undermodified hydroxywybutosine | OHyWy |
| Methylwybutosine | mimG |
| Peroxywybutosine | o2yW |
| Pre-Q0 base | preQ0base |
| Pre-Q1 base | preQ1base |
| Pseudouridine | Y |
| Queuosine | QtRNA |
| Undermodified hydroxywybutosine | OHyWx |
| Uridine | U |
| Uridine 5-oxyacetic acid | cmo5U |
| Uridine 5-oxyacetic acid methyl ester | mcmo5U |
| Wybutosine | yW |
| Wyosine | imG |

These modifications can be distinguished by any method that is known in the art, such as mass spectrometry, specific chemical reaction, or comparison with a standard synthetic product (e.g., comparison of retention time on LC), and optionally utilizing information that has been accumulated up to that point.

As used herein, “modification state”, in the context of a nucleic acid, refers to any state of a modification of a nucleic acid, including any item such as the presence/absence of modification, type of modification, position of modification (modified position on a base sequence, modified position on a chemical structure, etc.), ratio of modified nucleic acids, and modification ratio at a specific modified position. Since modification also includes forms in which the nucleic acid itself has changed, modification state also includes information on whether the nucleic acid itself has changed from a naturally occurring form.

As used herein, “methylation”, in the context of a nucleic acid, refers to methylation of any position of any type of nucleotide and is typically methylation of adenine (e.g., position 6; m6A, position 1; m1A) or methylation of cytosine (e.g., position 5; m5C, position 3; m3C). A detected modified site can be identified using a methodology that is known in the art. For example, each of m1A and m6A and m3C and m5C can be determined by chemical modifications. For example, it is possible to determine whether a behavior according to measurement by MALDI and chemical modification is correct by utilizing a standard synthetic RNA.

As used herein, a modification of a nucleic acid is intended to include modifications on a saccharide moiety and phosphoric acid moiety in addition to modifications on a base moiety of the nucleic acid. As used herein, a modification of a nucleic acid is also intended to include artificially introduced modifications in addition to naturally-occurring modifications. Examples of nucleic acids with a modified saccharide moiety include locked nucleic acids (LNA), ethylene nucleic acids such as 2′-O,4′-C-ethylene bridged nucleic acids (ENA), other bridged nucleic acids (BNA), hexitol nucleic acids (HNA), Amido-bridged nucleic acids (AmNA), morpholino nucleic acids, tricyclo-DNA (tcDNA), polyether nucleic acids (see, for example, U.S. Pat. No. 5,908,845), cyclohexene nucleic acids (CeNA), and the like. Examples of nucleic acids with a modified phosphoric acid moiety include nucleic acids with a phosphodiester bond replaced with a phosphothioate bond.

As used herein, “modified position on a base sequence” of a nucleic acid refers to a position where a modified base is present in the base sequence. For example, the modified position on the base sequence for a sequence of AAA(m6A)AA is the 4^{th} position.

As used herein, “modified position on a chemical structure” of a nucleic acid refers to a position where a modification is present within a nucleotide unit of the nucleic acid. For example, the modified position on the base sequence for a nucleic acid represented by AAA(m6A)AA is the 4^{th} position, and the modified position on a chemical structure is on N at position 6.

As used herein, “modification ratio” of a nucleic acid refers to the percentage representing the number of hits with at least one modification in a specific sequence among the number of hits with a specific sequence that have been detected. For example, if the specific sequence is AAAAA, and 100 hits of GGGGG, 70 hits of GGAAAAACC, 20 hits of

GGAAA(m6A)A, and 10 hits of GGAAA(m6A)(m6A)CC have been detected, the modification ratio of a nucleic acid is calculated with the sum of GGAAAAACC, GGAAA(m6A)A, and GGAAA(m6A)(m6A)CC, which is 100 hits, as the denominator and the sum of GGAAA(m6A)A and GGAAA(m6A)(m6A)CC that comprise a modified sequence, which is 30 hits, as the numerator, so that the modification ratio is 30%.

As used herein, “modification ratio at a modified position” of a nucleic acid refers to the percentage representing the number of hits with a modification at a specific modified position in a specific sequence (modified position on a base sequence or modified position on a chemical structure) among the number of hits with the specific sequence that have been detected. For example, if 100 hits of GGGGG, 70 hits of GGAAAAACC, 20 hits of GGAAA(m6A)A, and 10 hits of GGAAA(m6A)(m6A)CC have been detected, the modification ratio at the 4^{th} modified position on the base sequence of a nucleic acid with AAAAA is calculated in the same manner as the previous paragraph, which is 30% in view of the denominator of 70+20+10 hits (100 hits) and the numerator of 20+10 hits (30 hits), and the modification ratio at the 5^{th} modified position on the base sequence is calculated in the same manner as the previous paragraph, which is 10% in view of the denominator of 70+20+10 hits (100 hits) and the numerator of 10 hits.

As used herein, “tunneling current” refers to a current generated by an electron moving beyond the energy barrier.

As used herein, “pattern” of a tunneling current refers to a characteristic of the tunneling current expressed by any feature (e.g., current value (ampere), time, or the like) of the tunneling current.

As used herein, “measurement” is used in the meaning that is commonly used in the art, referring to determining the presence/absence, level, amount, or the like of a certain subject. Measurement includes quantitative as well as qualitative measurement. As used herein, “detection” is used in the meaning that is commonly used in the art, referring to investigating and finding a substance, component, or the like. “Identification” refers to an act of searching for where a certain subject belongs to from among known classifications that are associated therewith. When used in the field of chemistry, identification refers to determining the identity of a target subject as a chemical substance (e.g., determining a chemical structure). “Quantification” refers to determination of the amount of a target substance.

As used herein, the “amount” of an analyte in a sample generally refers to an absolute value reflecting the mass of the analyte that can be detected in a volume of sample. However, amount is also intended as a relative amount as compared to the amount of another analyte. For example, the amount of an analyte in a sample can be an amount that is greater than a control level or a normal level of an analyte that is generally present in a sample.

The term “about”, when used herein in relation to a quantitative measurement excluding measurement of the mass of an ion, refers to the indicated value plus or minus 10%. Even if “about” is not explicitly indicated, a value can be interpreted in the same manner as if the term “about” is used. Mass spectrometers can vary slightly in the determination of mass of a given analyte. The term “about” in relation to the mass of ions or the mass/charge ratio of ions refers to +/−0.5 atom mass unit.

As used herein, “subject” refers to a subject targeted for the analysis, diagnosis, detection, or the like of the present disclosure (e.g., food, organism such as a human or microorganism, cell, blood, or serum retrieved from an organism, or the like). In the case of the subject of a test or trial, it is referred to as test subject or trial subject or the like.

As used herein, “biomarker” is an indicator for evaluating a condition or action of a subject. Unless specifically noted otherwise, “biomarker” is also referred to as “marker” herein.

The detecting agent or detection means of the present disclosure can be a complex or complex molecule prepared by coupling, to a portion that is made detectable (e.g., antibody or the like), another substance (e.g., label or the like). As used herein, “complex” or “complex molecule” refers to any construct including two or more portions. For example, if one of the portions is a polypeptide, the other portion can be a polypeptide or other substances (e.g., substrate, saccharide, lipid, nucleic acid, other carbohydrate, or the like). The two or more portions constituting a complex herein can be bound by a covalent bond or other bonds (e.g., hydrogen bond, ion bond, hydrophobic interaction, van der Waals force, or the like). If two or more portions are polypeptides, the complex can be referred to as a chimeric polypeptide. Therefore, “complex” as used herein includes molecules prepared by linking a plurality of types of polypeptides, polynucleotides, lipids, saccharides, small molecules, or other molecules.

As used herein, “means” refers to anything which can be a tool for attaining a certain objective (e.g., detection, diagnosis, or therapy). As used herein, “means for selective recognition (detection)” especially refers to means which can recognize (detect) a certain subject differently from others.

As used herein, “mass spectrometry” or “MS” is used in the meaning that is commonly used in the art. This refers to an analytical approach for identifying a compound by its mass, referring to a technology for producing gaseous ions (ionization) from particles such as atoms, molecules, or clusters by some type of method, allowing the ions to move in a vacuum, and using electromagnetic force or the like or difference in the time of flight or the like to separate/detect the ions in accordance with the mass to charge ratio. MS refers to a method of filtering, detecting, and measuring ions based on mass to charge ratio, i.e., “m/z”. With the recent dramatic improvement in the detection sensitivity and mass resolution, the scope of application thereof has further broadened such that utility is found in many fields. Typically, a method exemplified in Clark J et al., Nat Methods. 2011 March; 8(3): 267-272.doi:10.1038/nmeth.1564. can be used. The MS technology generally includes: (1) ionizing a compound to form a charged compound; and (2) detecting the molecular weight of the charged compound to calculate the mass to charge ratio. A compound can be ionized and detected by suitable means. A “mass spectrometer” generally comprises an ionization apparatus, a mass spectrometer, and an ion detector. Generally, one or more molecules of interest is ionized.

The ion is then introduced into a mass spectrometer, where the ion follows a path in space that is dependent on mass (“m”) and charge (“z”) due to the combination of magnetic field and electric field. See, for example, Jurgen H, “Mass Spectrometry”, Maruzen Publishing (2014) for an outline of a mass spectrometer. Examples of mass spectrometers include magnetic field, electric field, quadrupole, time-of-flight mass spectrometers, and the like. Examples of ion detection in quantification include selective ion monitoring for selectively detecting only ions of interest, selective reaction monitoring (SRM) for selecting one of the ion types purified at the first mass spectrometry unit as a precursor ion and detecting a product ion generated by cleaving the precursor ion in the second mass spectrometry unit, and the like. In SRM, selectivity is increased, and noise is decreased, thus improving the signal/noise ratio.

As used herein, the term “resolution” broadly refers to the capability of measuring or identifying a subject with an apparatus or the like, and “resolution (FWHM)” (also known in the art as “m/Δm50%”) that is particularly used herein refers to the observed mass to charge ratio divided by the width of mass peak at 50% of the maximum height (full width at half maximum, “FWHM”). When simply denoted as “resolution” herein, this may refer to this specific resolution (FWHM). The qualitative and quantitative determination can be improved with higher resolution.

As used herein, “label” refers to an entity (e.g., substance, energy, electromagnetic wave, or the like) for distinguishing a molecule or substance of interest from others. Examples of such a labeling method include RI (radioisotope) method, stable isotope labeling, fluorescence method, biotin method, optical approaches utilizing Raman scattering, chemiluminescent method, and the like. When a plurality of markers of the present disclosure or agents or means for capturing the same are labeled by a fluorescence method, labeling is performed with fluorescent substances having different fluorescent emission maximum wavelengths. When a plurality of markers of the present disclosure or agents or means for capturing the same are labeled by an optical approach utilizing Raman scattering, labeling uses substances with different Raman scattering from each other. In the present disclosure, such a label can be utilized to modify a subject of interest so that the subject is detectable by detection means that is used. Such a modification is known in the art. Those skilled in the art can practice such a method as appropriate in accordance with the label and subject of interest.

As used herein, “diagnosis” refers to identifying various parameters associated with a condition (e.g., disease, disorder, or the like) in a subject or the like to determine the current or future state of such a condition.

The condition in the body can be investigated by using the method, apparatus, or system of the present disclosure. Such information can be used to select and determine various parameters of a formulation or method for the treatment or prevention to be administered, or condition in a subject, or the like. As used herein, “diagnosis” when narrowly defined refers to diagnosis of the current state, but when broadly defined includes “early diagnosis”, “predictive diagnosis”, “prediagnosis”, and the like. Since the diagnostic method of the present disclosure in principle can utilize what comes out from a body and can be conducted away from a medical practitioner such as a physician, the present disclosure is industrially useful. In order to clarify that the method can be conducted away from a medical practitioner such as a physician, the term as used herein may be particularly called “assisting” “predictive diagnosis, prediagnosis, or diagnosis”. The technology of the present disclosure can be applied to such a diagnostic technology.

As used herein, “therapy” refers to the prevention of exacerbation, preferably maintaining of the current condition, more preferably alleviation, and still more preferably disappearance of a condition (e.g., disease or disorder) in case where such a condition appeared, including being capable of exerting a prophylactic effect or an effect of improving a condition of a patient or one or more symptoms accompanying the condition. Preliminary diagnosis with suitable therapy is referred to as “companion therapy” and a diagnostic agent therefor may be referred to as “companion diagnostic agent”. If a modification of RNA can be identified using the technology of the present disclosure, the modification can be associated with a specific condition, so that can be useful in such companion therapy or companion diagnosis.

The term “prognosis” as used herein refers to prediction of the possibility of death due to a disease or disorder such as cancer or progression thereof. A prognostic factor is a variable related to the natural course of a disease or disorder, which affects the rate of recurrence in a patient who has developed the disease or disorder. Examples of clinical indicators associated with exacerbation in prognosis include any cell indicator used in the present disclosure. A prognostic factor is often used to classify patients into subgroups with different pathological conditions. If a modification of RNA can be identified using the technology of the present disclosure, the modification can be associated with a specific disease condition, so that this can be useful as a technology for providing a prognostic factor.

As used herein, “detector” broadly refers to any instrument that can detect or test a subject of interest. As used herein, “diagnostic drug” broadly refers to any agent capable of diagnosing a condition of interest (including, for example, medical conditions such as cancer and senescence as well as other conditions, species classification, and the like).

As used herein, “kit” refers to a unit providing portions to be provided (e.g., test drug, diagnostic drug, therapeutic drug, reagent, label, descriptions, and the like), which is generally provided in two or more separate sections. This form of a kit is preferred when a composition that should not be provided in a mixed state and is preferably mixed immediately before use for safety or other reasons is intended to be provided. Such a kit advantageously comprises an instruction or descriptions describing how the provided portions (e.g., test drug, diagnostic drug, therapeutic drug, reagent, label, and the like) are used or handled. When the kit is used herein as a reagent kit, the kit comprises instructions describing how to use a test drug, diagnostic drug, therapeutic drug, reagent, label, and the like. When combined with a testing instrument, diagnostic instrument, or the like, a “kit” can be referred to as a “system”.

As used herein, “program” is used in the meaning that is commonly used in the art. A program describes the processing to be performed by a computer in order, and is considered as a “product” under the Japanese Patent Law. A program may also be referred to as a “program product” in order to make it clear that the program is perceivable or tangible. All computers operate in accordance with a program. Programs are expressed as data in modern computers, and can be stored in a recording medium or a storage device or provided from the cloud.

As used herein, “recording medium” is a medium for storing a program for executing the method described herein. A recording medium can be anything, as long as the medium can record a program, is computer-readable, and thus can cause another instrument such as a computer to execute or implement a program that has been read out. For example, a recording medium can be, but is not limited to, a ROM or HDD or a magnetic disk that can be stored internally, or an external storage device such as flash memory such as a USB memory.

As used herein, “system” refers to a configuration that executes the method or program of the present disclosure. A system fundamentally refers to a system or organization for executing an objective, wherein a plurality of elements are systematically configured to affect one another, and a plurality of various apparatuses are optionally configured to communicate with one another. In the field of computers, system refers to the entire configuration of the hardware, software, OS, network and the like.

As used herein, “agent” is used broadly and may be any substance or other elements (e.g., light, radiation, heat, electricity, and other forms of energy) as long as the intended objective can be achieved (“inhibiting agent”, for example, can be considered an agent that “inhibits” a target of interest). Examples of such a substance include, but are not limited to, protein, polypeptide, oligopeptide, peptide, polynucleotide, oligonucleotide, nucleotide, nucleic acid (including, for example, DNAs such as cDNA and genomic DNA and RNAs such as mRNA), polysaccharide, oligosaccharide, lipid, organic small molecule (e.g., hormone, ligand, information transmitting substance, organic small molecule, molecule synthesized by combinatorial chemistry, small molecule that can be used as medicine (e.g., small molecule ligand, etc.), and the like), and composite molecule thereof. Typical examples of an agent specific to a polynucleotide include, but are not limited to, a polynucleotide having complementarity with a certain sequence homology (e.g., 70% or greater sequence identity) to the sequence of the polynucleotide, polypeptide such as a transcription factor that binds to a promoter region, and the like. Typical examples of an agent specific to a polypeptide include, but are not limited to, an antibody directed specifically to the polypeptide or a derivative or analog thereof (e.g., single chain antibody), a specific ligand or receptor when the polypeptide is a receptor or ligand, a substrate when the polypeptide is an enzyme, and the like.

### Preferred Embodiments

The preferred embodiments are described hereinafter. It is understood that the embodiments are exemplification of the present disclosure, so that the scope of the present disclosure is not limited to such preferred embodiments. It is understood that those skilled in the art can also refer to the following preferred embodiments to readily make modifications or changes within the scope of the present disclosure. It is understood that one or more of any of the embodiments can be combined. Descriptions of underlying technologies used in the present disclosure are also provided hereinafter.

(Electrode Pair)

As used herein, “electrode pair” is used in the meaning that is commonly used in the art, generally referring to a pair of electrodes.

In the present disclosure, a tunneling current that is generated when a subject such as a microRNA passes between an electrode pair is detected to take measurement on the subject. The distance between the electrode pair can be important for suitably generating a tunneling current. If the distance between an electrode pair is much greater than the molecular diameter of each nucleotide constituting a microRNA, it can be difficult for a tunneling current to flow between the electrode pair, or two or more microRNAs can simultaneously enter between the electrode pair. Meanwhile, if the distance between an electrode pair is much less than the molecular diameter of each nucleotide constituting a microRNA, the microRNA would not be able to enter between the electrode pair. If the distance between an electrode pair is much greater or much less than the molecular diameter of a nucleotide constituting a microRNA in this manner, it can be difficult to detect a pulse originating from a tunneling current via each single nucleotide molecule constituting the microRNA. Accordingly, the distance between an electrode pair is preferably somewhat shorter than, equal to, or somewhat greater than the molecular diameter of a nucleotide constituting a microRNA. For example, the distance between an electrode pair is 0.5- to 2-fold in length, preferably 1- to 1.5-fold in length, and more preferably 1- to 1.2-fold in length relative to the molecular diameter of a nucleotide. For example, the molecular diameter of a nucleotide in a form of a monophosphate is about 1 nm, so that in one embodiment, the distance between an electrode pair can be set to, for example, 0.5 nm to 2 nm, 1 nm to 1.5 nm, or 1 nm to 1.2 nm based on such a molecular diameter.

In one embodiment, the distance between an electrode pair can be maintained at a constant distance during measurement, i.e., can be controlled so that the distance between the electrode pair does not change during measurement. For example, the ratio of change in the distance between an electrode pair during measurement can be 5% or less, 2% or less, 1% or less, 0.1% or less, 0.01% or less, or 0.001% or less. By maintaining a constant distance between an electrode pair, the accuracy of identifying the base sequence and/or modification state of a microRNA can be improved.

The electrode pair used in the present disclosure can be prepared by any suitable method. For example, an electrode pair can be prepared by using the known nanofabricated mechanically-controllable break junctions. Nanofabricated mechanically-controllable break junctions is an excellent method that can control the distance between electrodes with excellent mechanical stability with the resolution of 1 picometer or less. Preparation methods of an electrode pair using nanofabricated mechanically-controllable break junctions are described in, for example, J. M. van Ruitenbeek, A. Alvarez, I. Pineyro, C. Grahmann, P. Joyez, M. H. Devoret, D. Esteve, C. Urbina, Rev. Sci. Instrum. 67, 108 (1996) or M. Tsutsui, K. Shoji, M. Taniguchi, T. Kawai, Nano Lett. 8, 345 (2008). As the material of each electrode in an electrode pair, any conductive material can be used, and metal (e.g., gold, etc.) for example can be used. A specific exemplary procedure for preparing an electrode pair is described, for example, in the Examples. A constant distance between electrodes can also be readily maintained in a solution for nanogap electrodes prepared by mechanically breaking a fine metal wire prepared by micromachining or nanogap electrodes on a substrate prepared by micromachining through a piezo actuator feedback method.

(Tunneling Current Measurement)

A tunneling current can be measured by passing a microRNA between an electrode pair. For example, a microRNA can be passed between an electrode pair by allowing a fluid comprising the microRNA to flow so as to pass through an apparatus comprising the electrode pair. As the fluid, a medium that would result in dispersion of microRNA can be used. In one embodiment, a medium that does not generate a tunneling current is used. Examples thereof include, but are not limited to, ultrapure water. In one embodiment, the concentration of microRNAs in a fluid can be 0.0001 to 100 μM (μmol/L) such as at least 0.0001 μM, at least 0.0002 μM, at least 0.0005 μM, at least 0.001 μM, at least 0.002 μM, at least 0.005 μM, at least 0.01 μM, at least 0.02 μM, at least 0.05 μM, at least 0.1 μM, at least 0.2 μM, at least 0.5 μM, at least 1 μM, at least 2 μM, at least 5 μM, or at least 10 μM, and at most 100 μM, at most 50 μM, at most 20 μM, at most 10 μM, at most 5 μM, at most 2 μM, at most 1 μM, at most 0.5 μM, at most 0.2 μM, at most 0.1 μM, at most 0.05 μM, at most 0.02 μM, or at most 0.01 μM (any combination of an upper limit and a lower limit is intended, as long as there is no contradiction). Even if the concentration of microRNAs in a sample is unknown, the microRNA can be first measured by some type of a method to obtain information on the approximate content and concentration, and then re-measured by concentrating and diluting to a concentration that is suitable for measuring a tunneling current. If at least one molecule of microRNA is contained in a fluid, the base sequence and/or modification state can be analyzed.

By applying a voltage between an electrode pair, a tunneling current is generated between the electrode pair when a microRNA passes between the electrode pair. In one embodiment, the applied voltage can be, for example, 0.1 V to 1 V, such as 0.25 V to 0.75 V, but the voltage is not particularly limited. A method of applying a voltage between an electrode pair is not particularly limited. For example, a voltage (e.g., bias voltage) can be applied between an electrode pair by connecting a known power source to the electrode pair.

In one embodiment, a microRNA of interest can be physically, chemically, or biologically treated in advance prior to measurement. Preliminary treatment can attain an effect such as improvement in the sensitivity, accuracy, and/or precision of measurement on the microRNA of interest, further differentiation in a modification state, improvement in quantification in a comparison between samples, control of the direction of movement in a solution, or orientation of a microRNA that enters an electrode pair (e.g., preferential entrance from the 3′). In one embodiment, an agent used in preliminary treatment can be designed to introduce a group into an amine moiety on a base of a microRNA, or a phosphoric acid group or hydroxyl group at the terminus.

While the specific method of passing a microRNA between an electrode pair is not particularly limited, a microRNA can be moved by, for example, thermal diffusion (e.g., Brownian motion), AC voltage, or the like and passed between an electrode pair by the movement. In one preferred embodiment, a microRNA can be moved by thermal diffusion and passed between an electrode pair by the movement. By doing so, the microRNA can stay between the electrode pair for an extended period of time, so that more information on the microRNA can be obtained.

The temperature for thermal diffusion of a microRNA is not particularly limited and can be appropriately determined. For example, a temperature such as 5° C. to 70° C. or 20° C. to 50° C. can be used.

Unlike conventional technologies, in the present disclosure, an electrode having a pore formed with a protein is not needed. Thus, the electrode would not lose its function even after thermal diffusion of a microRNA at a high temperature. If a microRNA is thermally diffused at a high temperature, intermolecular/intramolecular interaction (e.g., hydrogen bond) of microRNAs can be prevented, and formation of a complementary strand pair can be prevented, so that the base sequence and/or modification state of the microRNA can be more accurately identified.

When a microRNA passes between an electrode pair while a voltage is applied between the electrode pair, a tunneling current due to a nucleotide constituting the microRNA is generated between the electrode pair. The mechanism by which a tunneling current is generated is described below.

When a microRNA enters between an electrode pair, the first nucleotide, which is one of nucleotides constituting the microRNA, is initially captured between the electrode pair, and a tunneling current due to the first nucleotide is generated between the electrode pair. In this regard, the first nucleotide can be a nucleotide at the 5′ terminus of a polynucleotide, a nucleotide at the 3′ terminus of a polynucleotide, or a nucleotide that is present between the 5′ terminus and the 3′ terminus.

Subsequently, after the first nucleotide passes between the electrode pair, a second nucleotide is captured between the electrodes, and a tunneling current due to the second nucleotide is generated between the electrode pair. In this regard, the second nucleotide can be a nucleotide that is adjacent to the first nucleotide, or a nucleotide that is not adjacent to the first nucleotide. The position of the second nucleotide can be on the 5′ terminus side or on the 3′ terminus side of the first nucleotide.

In this manner, tunneling currents due to a plurality of nucleotides of a microRNA are generated between an electrode pair. When a microRNA passes between an electrode pair, the tunneling current that has been generated between the electrode pair disappears.

A tunneling current generated between an electrode pair can be measured using a known ammeter. A signal of a tunneling current can be amplified by using a current amplifier. Since a weak tunneling current value can be amplified by using a current amplifier, a tunneling current can be measured at a high sensitivity. Any current amplifier can be used. Examples thereof include a commercially available variable high speed current amplifier (Femto, Catalog No.: DHPCA-100).

A tunneling current can be affected by the distance between electrodes, concentration of microRNA in a solution, shape of electrodes, voltage between an electrode pair, or the like. For this reason, data (e.g., feature of tunneling current) can be suitably adjusted when comparing with or referring to a result under a different measurement condition.

The same nucleotides can generate tunneling currents having different features (e.g., different peak heights). For example, peaks with different heights can manifest due to a change in the distance between an electrode and a nucleotide in view of the movement of the nucleotide. Specifically, if the distance between a nucleotide and an electrode is shortened, a tunneling current is more readily generated. Thus, a current value of a tunneling current increases to result in manifestation of a higher peak. For this reason, in one embodiment, wide-ranging features (e.g., peak heights in a certain range) and/or a combination of different types of features is used in order to identify the sequence and/or modification state of a microRNA.

(Feature of Tunneling Current)

Any measured feature (e.g., peak height, peak width, peak frequency, peak shape, combination thereof, etc.) of a tunneling current can be used to identify the base sequence and/or modification state of a microRNA. In one embodiment, a pattern of a tunneling current can be expressed by these features or combination of features. In one embodiment, the tunneling current that is generated when a microRNA passes between an electrode pair can itself be used for identification. In one embodiment, a pulse of a tunneling current that is generated when a microRNA passes between an electrode pair can be used for identification.

For identification, a current value of a tunneling current can be used, or conductance of a tunneling current can be used in place of a current value. Conductance can be calculated by dividing a current value of a tunneling current by a voltage applied to an electrode pair. A profile under a uniform baseline can be obtained by using conductance, even if the value of voltage applied between an electrode pair varies for each measurement. If the value of voltage applied between an electrode pair is held constant for each measurement, a current value and conductance of a tunneling current can be handled in the same manner.

In one embodiment, a plurality of pulses, one pulse or no pulse may be detected for a single base in a microRNA molecule that passes through. While the number of pulses detected for each base in a microRNA molecule is not particularly limited, a greater number can result in identification of the type and/or modification state of the base with a higher accuracy and/or precision. The number of pulses detected can be greater with a longer measurement time. In one embodiment, the mean measurement time for each nucleotide can be, for example, about 5 milliseconds, about 10 milliseconds, about 20 milliseconds, about 50 milliseconds, about 100 milliseconds, about 200 milliseconds, about 500 milliseconds, about 1000 milliseconds, about 2000 milliseconds, about 5000 milliseconds, or about 10000 milliseconds.

A pulse of a tunneling current can be detected by measuring the tunneling current flowing between an electrode pair and determining whether a current value of the tunneling current exceeds a baseline level over time. Any timeframe including a current value of a tunneling current that exceeds the baseline level can be detected as a pulse. For example, the point at which a tunneling current exceeds the baseline level and the point at which the tunneling current reverts to the base level can be identified, and a signal between these two points can be detected as a pulse of a tunneling current due to a nucleotide. A single pulse may be associated with one or more nucleotides, or one or more pulses may be associated with a single nucleotide.

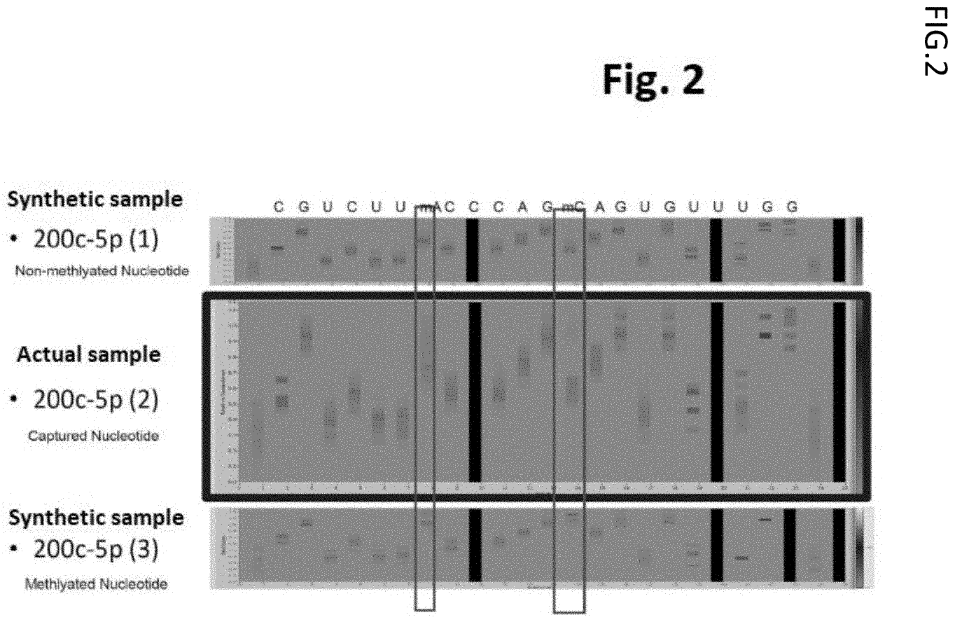

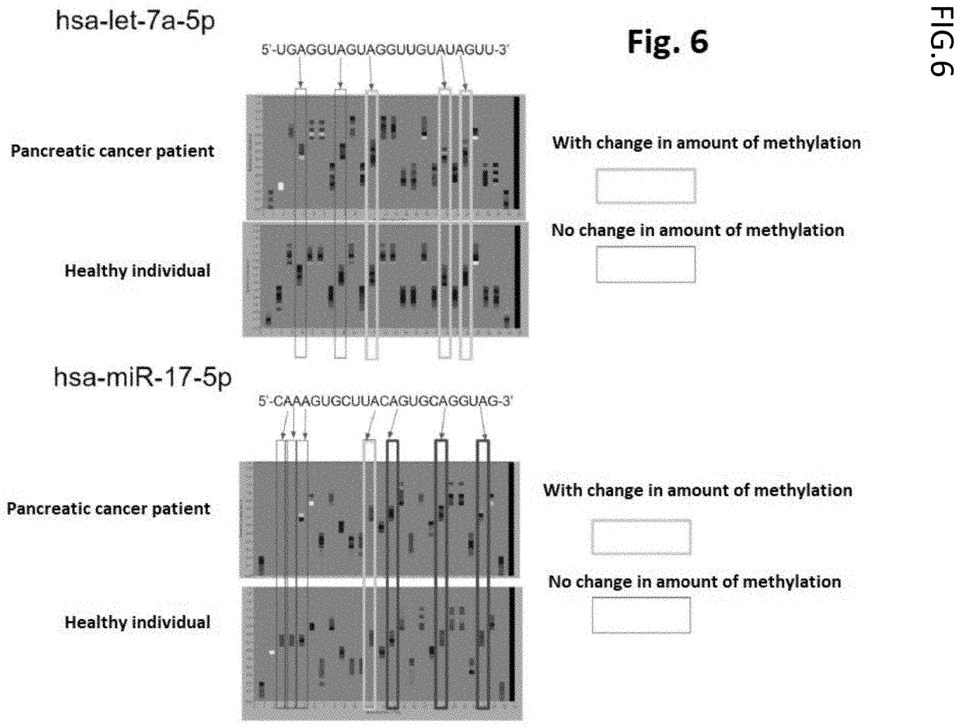

FIGS. 6(A) and 6(B) show an example of a pulse of a tunneling current. Any feature of each pulse or a combination of pulses can be extracted from a graph showing measured current values of a tunneling current and tunneling current measurement times as shown in FIG. 2 for use in identification. Examples of such a feature that can be used include, but are not limited to, the magnitude of current, frequency of pulses per unit time, pulse duration, pulse shape, and the like. In a specific embodiment, the maximum current value (Ip) of a pulse and/or pulse duration (tp) can be used for identification.

In one embodiment, after a current value exceeding the baseline level is detected and a tunneling current starts to be generated on the first nucleotide, generation of a tunneling current may start on a second nucleotide before the current value reverts back to the baseline level in some cases (e.g., FIG. 6(C)). In such a case, it is understood that the first nucleotide and the second nucleotide are highly likely to be contiguous nucleotides in a molecule. Since the current value does not revert back to the baseline level, this can be counted as a single pulse for a plurality of nucleotides, and may also be counted as a plurality of pulses depending on the pulse feature (e.g., current value and pulse duration).

In one embodiment, the maximum current value of each pulse can be calculated by subtracting the baseline level from a current value of the highest peak from each pulse in the results of measuring a tunneling current for a certain nucleotide. The mode can be computed by performing statistical analysis on each of the maximum current values that have been calculated. For example, a histogram showing the relationship between the value of the maximum current value and the number of pulses with said value is created in order to find the mode. The created histogram is then fitted to a given function. The mode can then be computed by finding the peak value from the fitted function.

Examples of functions used in fitting include the Gaussian function and Poisson function. Since the mode can be a value unique to each nucleotide under the same measurement condition and/or same environment, the mode can be used as an indicator for identifying a nucleotide constituting a polynucleotide.

Since the mode of maximum current values has a distribution, the mode can be used as a mode at a single point or a distribution of the mode. For example, the distribution of the mode of maximum current values of the first nucleotide can be compared to the maximum current values or the distribution of the mode of the maximum current values of the second nucleotide to determine the similarity between the first nucleotide and the second nucleotide (e.g., the probability of being the same nucleotide or the possibility of being in a relationship of a modified nucleotide and an unmodified nucleotide).

(Identification of Base Sequence and/or Modification)

In the present disclosure, the base sequence and/or modification information for a microRNA of interest is identified based on a result of measuring a tunneling current of the microRNA. In one embodiment, both the base sequence and modification information for a microRNA are identified. In one embodiment, a modified position on a base sequence of a microRNA is identified. In one embodiment, a modified position on a chemical structure of a microRNA is identified. In one embodiment, a modification ratio of a microRNA is identified. In one embodiment, a modification ratio at a specific modified position of a microRNA is identified. In one embodiment, information on a modification of a nucleotide itself of a microRNA is identified. Any type of modification can be identified. In accordance with the present disclosure, any type of microRNA can be identified by a pattern of a tunneling current. In one embodiment, for example, methylation of a microRNA is identified. Any suitable reference information other than results of measuring a tunneling current of a microRNA of interest can be referenced for identification.

Not all nucleotides in the microRNA measured need to be identified. For example, it can be sufficient to identify only a specific base sequence and/or modification state at a specific position. Identification results may be outputted with the probability of being a specific base sequence and/or modification state. In one embodiment, chronological signal data for conductance values is obtained and assembled for each microRNA sequence to create a histogram of conductance values, which can be used for identifying the type of microRNA and/or associating a signal with a nucleotide at a specific position.

In one embodiment, each of one or more nucleotides in the measured microRNA can be identified. In one embodiment, a partial structure in the measured microRNA can be identified. In one embodiment, the entire measured microRNA molecule can be identified. For example, it is possible to identify whether two measurement results are for the same microRNA molecule by comparing tunneling currents of the entire microRNA molecules with each other.

In one embodiment, reference information for identifying each nucleotide is created by measuring tunneling currents of various modified nucleotides and unmodified nucleotides and obtaining a feature (e.g., maximum current value or the like) of a pulse. In this regard, modified nucleotides and unmodified nucleotides can be measured as a mononucleotide or measured as a nucleotide incorporated into a polynucleotide (e.g., polynucleotide having the same structure except for the modified or unmodified nucleotide or interest). For example, such reference information can be obtained by measuring a synthesized microRNA, or by measuring a microRNA that has been concentrated by using a specific modification specific antibody.

In one embodiment, reference information for identifying each nucleotide can be created from a value computed based on the structure of a modified and/or unmodified nucleotide, or created by combining a computed value and measurement value. For example, such a computed value can be obtained by computing the highest occupied molecular orbital (HOMO) based on density functional theory, based on the structure of a modified and/or unmodified nucleotide.

In one embodiment, the conductance values of a nucleotide and modified nucleotide can be measured in monomer data and the range of the conductance values of the nucleotide and modified nucleotide in a nucleic acid sequence can be set while taking into consideration the peak position and shape of a histogram. For example, the conductance values of adenine and methylated adenine in a nucleic acid sequence can be set in a range of 0.60 to 0.8 and 0.75 to 0.90, respectively, based on the conductance values of adenine and methylated adenine in monomer data (0.7 and 0.8, respectively). For each signal corresponding to a position on a nucleic acid of interest, whether a nucleotide is modified can be determined by using a probability density from a Gaussian function or the like as an indicator. In one embodiment, the number of adenine and the number of methylated adenine can be counted based on such a determination result, and the methylation ratio (amount) can be computed as methylated adenine count/(adenine count +methylated adenine count). If the difference in methylation ratios between results of measuring microRNAs obtained from a sample of interest and a reference sample exceeds a specific value, such as about 1 to 10000%, about 1%, about 2%, about 3%, about 4%, about 5%, about 7%, about 10%, about 10%, about 20%, about 50%, about 70%, about 100%, about 200%, about 500%, about 700%, about 1000%, about 2000%, about 5000%, or about 10000%, the sample of interest can be identified as being in a medical or biological condition of interest.

In one embodiment, the sequence and/or modification state of a microRNA contained in a sample can be identified by using a result of measuring a sample that is the same or similar to the sample measured with a tunneling current by another measuring means as reference information. Since a measured microRNA can be retrieved without decomposition in tunneling current measurement, the retrieved microRNA can be subjected to another analysis means. In one embodiment, a dispensed sample from the same mixture sample can be subjected to tunneling current measurement, and another dispensed sample from the same sample can be subjected to another analysis means.

In one embodiment, a result of tunneling current measurement and a result of mass spectrometry can be combined. A base sequence and/or modification state (e.g., position and presence/absence) of a microRNA contained in a sample can be identified at a high throughput by mass spectrometry. In mass spectrometry, even if the type of modification (e.g., monomethylation of adenine) is found, it can be difficult to detect the difference in the modified position on a chemical structure as a difference in the mass number. The modified position on a chemical structure is generally identified in combination with another information such as derivatization through chemical processing of a sample. Meanwhile, in tunneling current measurement, the difference in the modified position on a chemical structure can be detected as a difference in tunneling current. For this reason, combining tunneling current measurement with mass spectrometry can complement information of each other, and reference information for identifying the base sequence and/or modification state of a microRNA based on the tunneling current measurement can be collected at a high throughput. For example, if it is found that a base at a certain position on a microRNA is replaced with a modified base (having a specific difference in mass) as a result of mass spectrometry, a result of tunneling current measurement for the base position can be associated with modification information. In this regard, such modification information (e.g., information on the modified position on a chemical structure) can be corroborated by, for example, collation with known information on synthetic microRNAs.

In one embodiment, examples of mass spectrometers that can be used include magnetic field, electric field, quadrupole, and time-of-flight (TCF) mass spectrometers, and the like. Mass spectrometry can be combined with any ionization method. Examples of ionization method that can be used in the present disclosure include, but are not limited to, electron ionization (EI), chemical ionization (CI), fast atom bombardment (FAB), matrix-assisted laser desorption/ionization (MALDI), and electrospray ionization (ESI). ESI can be combined with liquid chromatography, supercritical chromatography, or the like. A plurality of types of RNAs can be measured while being separated by chromatography. Examples of columns that can be used in chromatography include hydrophilic interaction chromatography (HILIC) columns, reverse phase (RP) chromatography columns, and the like.

For MALDI, a sample is premixed with a substance (coating agent) that is readily ionized with a laser beam as a matrix, and is placed at a spot (anchor position) on a target plate. Irradiation thereof with a laser beam results in ionization. Examples of coating agent that can be used in the present disclosure include, but are not limited to, 3-HPA (3-hydroxypicolinic acid), DHC (diammonium hydrogen citrate), CHCA (a-cyano-4-hydroxycinnamic acid), and the like.

In one embodiment, a modification state of an RNA (e.g., presence/absence of a modification, modification location, number of modifications, reliability of a modification, or the like) can be identified based on a measurement value for an unfragmented ion (parental ion) and/or fragmented ion (daughter ion). In one embodiment, a modification state (e.g., amount of modification or the like) of an RNA can be identified by comparison with a control molecule (e.g., stable isotope labeled nucleic acid, unmodified nucleic acid, the other nucleic acid of a pair forming a complementary double strand, or the like). Mass spectrometry data can be converted into an RNA modification state by processing with any software. Examples of such software include, but are not limited to, DNA methylation analysis system MassARRAY© EpiTYPER (Sequenom).

Once a modification state can be associated with a measurement value of a tunneling current, the modification state (e.g., modified position on a chemical structure) can be identified just from tunneling current sequence measurement thereafter. In doing so, a specific nucleic acid sequence and modification state can be combined to identify the sequence and modification information thereof at once.

Any suitable method can be selected to substantiate the type of modification. For example, radiation emitted from a radioactive atom contained in a moiety constituting the modification (e.g., methyl moiety) can be checked, a bond of a molecule that specifically binds to a modification (e.g., modification specific antibody) can be measured (e.g., measurement of fluorescence), or a reaction product generated by a reaction with a molecule that specifically reacts with a modification can be measured (e.g., measurement of light which is a reaction product, detection of a biotin derivative generated by a reaction with streptavidin, etc.).

Identification of the base sequence and modification information on a microRNA based on results of measuring a tunneling current according to the present disclosure is, for example, shown with the following specific examples, but any of the examples is not limiting.

*For example, a nucleotide at a position predicted to be prone to a certain modification can be weighted so that the nucleotide is identified as a modified nucleotide at a higher probability.
*For example, a portion of the base sequence of a measured microRNA can be identified based on a result of tunneling current measurement, and if the reliability for some of the bases is low, microRNA candidates having the base sequence that was able to be identified are selected based on information on the organism that is the origin of the sample from which the microRNA was obtained, and the bases with low reliability can be identified from more limited choices.
*For example, if there is a nucleotide that cannot be identified with high accuracy as a result of interpreting the results of tunneling current measurement based on existing reference information, the nucleotide can be deemed as a modified nucleotide that is not within the existing reference information.
*For example, when identifying the modification ratio at the specific modified position, if a specific threshold value is not met, the presence/absence of a specific modification at a specific modified position does not need to be counted as a hit for neither presence nor absence of a modification.

In this manner, it is possible to accumulate reference information for identifying some or the entire base sequence and/or modification information on a measured microRNA based on the obtained results of measuring a tunneling current. Further, any modification, deletion, and/or addition can be made to the accumulated reference information.

In one aspect, the present disclosure provides a database constructed with reference information accumulated for identifying the base sequence and/or modification information on a measured microRNA based on a result of tunneling current measurement.

(Analysis Using the Base Sequence and/or Modification Information on a microRNA)

In one aspect, the present disclosure provides a method of analyzing a condition of a subject based on the base sequence and/or modification information on a microRNA. In another aspect, the present disclosure provides a method comprising associating the base sequence and/or modification information on a microRNA with a condition of a subject. In these methods, the base sequence and/or modification information on a microRNA can be identified by any method described above based on a result of tunneling current measurement.

In one embodiment, a microRNA is present in a sample. In a specific embodiment, a sample is derived from a subject. The Examples of the subject include, but are not limited to, mammals (e.g., human, chimpanzee, monkey, mouse, rat, rabbit, dog, horse, pig, cat, and the like), microorganisms (e.g., pathogen, microorganism used for fermentation, microbes such as E. coli, parasite, fungus, virus (e.g., RNA virus such as coronavirus), and the like), edible organisms (avian, fish, reptile, fungus, plant, and the like), organisms raised as pets, and bioindicator organisms. In one embodiment, a sample is derived from a subject who has, or has the potential to have, a specific condition. In one embodiment, examples of the specific condition include, but are not limited to, disease, age, sex, race, familial lineage, medical history, treatment history, status of smoking, status of drinking, occupation, information on living environment, and the like. In one embodiment, a sample is an organ, tissue, cell (e.g., circulating tumor cell (CTC) or the like), blood (e.g., plasma, serum, or the like), epidermis of the mucous membrane (e.g., in the oral cavity, nasal cavity, ear cavity, vagina, or the like), epidermis of the skin, biological secretion (e.g., saliva, nasal mucus, sweat, tear, urine, bile, or the like), stool, epidermal microorganism or a portion thereof obtained from a subject. In one embodiment, a sample is a cultured cell (e.g., organoid based on a cell obtained from a subject, specific cell strain, or the like). In one embodiment, a sample is food or a portion thereof, or a microorganism on food.

In one embodiment, a microRNA may or may not be purified in advance for measurement on the microRNA. As used herein, a “purified” substance or a biological agent (e.g., microRNA such as a genetic marker, protein, or the like) refers to a substance or biological agent with at least a part of an agent naturally accompanying it removed. Therefore, the purity of a biological agent in a purified biological agent is higher than the normal state of the biological agent (e.g., concentrated). As used herein, the term “purified” means that preferably at least 75% by weight, more preferably at least 85% by weight, still more preferably at least 95% by weight, and most preferably at least 98% by weight of the same type of biological agents are present. A substance used in the present disclosure is preferably a “purified” substance. As used herein, “isolated” refers to a state resulting from removal of at least one substance from a naturally-occurring state. For example, retrieval of a specific microRNA from whole microRNA can be considered isolation. Thus, the microRNA used herein can be an isolated microRNA. In one embodiment, all types of microRNA can be purified from other components without distinguishing therebetween. In one embodiment, a microRNA having a sequence of interest (one or more types) can be purified from other components. In one embodiment, a microRNA having a modification (one or more types) can be purified from other components. In one embodiment, a microRNA having a methylation modification can be purified from other components.

Any known approach can be used for purification of microRNAs. In one embodiment, a microRNA of interest can be purified by treating with a DNA degrading enzyme and then purifying a nucleic acid molecule. A plurality of types of microRNAs can be purified separately or in parallel or in a mixed state. In one embodiment, 1, 2, 3, 3, 4, 5, 10, 15, 20, 25, 30, 35, 40, 45, 50, 75, 100, 150, 200, 300, 400, 500, 750, 1000, 1500, 2000, 2500, or 3000 types of RNAs can be purified in parallel (e.g., by using a sequence specific

RNA capturing molecule that is bound to a carrier). In one embodiment, a microRNA having a sequence of interest can be purified using a nucleic acid molecule (e.g., DNA and RNA) that is at least partially complementary to the sequence of interest, wherein the complementary nucleic acid molecule can comprise any portion for purification. Examples of any portion for purification include, but are not limited to, carriers such as beads (can be magnetic as needed), one of the pair molecules that bind to each other such as biotin and streptavidin, a portion that allows pair molecules binding to each other to bind (e.g., alkyne moiety in click chemistry), antibody recognition moiety, and the like. In one embodiment, a microRNA of interest can be purified by using a specific binding molecule (e.g., antibody). In one embodiment, a microRNA of interest can be purified using a binding molecule (e.g., antibody) that is specific to an RNA modification (e.g., methylation). In one embodiment, a microRNA of interest can be purified using a binding molecule (e.g., antibody) that is specific to a specific sequence.

In one embodiment, a modification in a modified microRNA is an artificially introduced modification. Examples of artificially introduced modifications include, but are not limited to, modifications introduced by chemical synthesis. This also includes modifications that are generated by an agent binding to an RNA in an organism (including viruses) when the organism is treated with the agent. For example, treatment of an organism with an agent (e.g., anticancer agent) that chemically and directly interacts with a nucleic acid in the organism can result in a modified RNA into which a moiety derived from the agent is introduced. The method of the present disclosure can readily identify a nucleic acid that is highly likely to be introduced with such an artificial modification (type, location, or the like). A nucleic acid that is highly likely to be introduced with such an artificial modification can be useful as an indicator, biomarker, or the like for research and development of agents.

In one embodiment, a microRNA of interest can be purified by purifying an organelle (e.g., exosome). In one embodiment, an organelle (e.g., exosome) can be purified by centrifugation. In one embodiment, a microRNA of interest can be purified by using a molecule (e.g., antibody) binding to a molecule in an organelle (e.g., purify an exosome using an anti-CD63 antibody).

Various conditions can be analyzed by using the base sequence and/or modification information for a microRNA. In one embodiment, a medical condition or a biological condition of a subject is analyzed using the obtained base sequence and/or modification information for a microRNA. Examples of the medical condition or biological condition of a subject include, but are not limited to, a disease, senescence, immunological condition (e.g., intestinal tract immunity, systemic immunity, and the like), cell differentiation condition, responsiveness to an agent or treatment, and a condition of a microorganism (e.g., enterobacteria, epidermal bacteria, or the like) of a subject. Examples of diseases that can be analyzed by the present disclosure include, but are not limited to, a cranial nerve disease, pollution disease, disease in pediatric surgery, fungal disease, specific disease, infections, cancer (malignant tumor), gastrointestinal disease (including inflammatory bowel disease), neurodegenerative disease, allergic disease, parasitic disease, infectious disease of an animal, urinary tract tumor, various syndromes, respiratory disease, mammary gland tumor, personality disorder, skin disease, sexually transmitted disease, dental disease, psychiatric disease, renal urinary disease, ophthalmic disease, food poisoning, intermediate host for Gymnosporangium, hepatitis, cardiovascular disease, rare disease, connective tissue disease, symptom, zoonosis, paraphilia, immune disease (including intestinal tract immunity), congenital disease, developmental disorder, skin rash, congenital heart disease, regional disease name, phobia, viral infection, male reproductive system disease, animal disease, fish disease, proliferative disease, polyp, periodontal disease, mammary gland disease, genetic disease, hematological disease, endocrine metabolic disease, gynecological disease, disease causing fever and rash, soft tissue tumors, plant disease, and the like. Examples of diseases that can be particularly suitably analyzed in the present disclosure include, but are not limited to, cancer, inflammatory bowel disease, Alzheimer's or angiopathic dementia, borderline mental illness, dilated cardiomyopathy, hypertrophic cardiomyopathy, heart failure (including nonobvious mild heart failure), heart disease (e.g., including those that are fatal, inducing sudden death due to arrhythmia), and the like. These diseases can affect the modification state of an RNA via specific metabolism of a cell. Examples of a condition of a microorganism of a subject include, but are not limited to, a condition that can be a public health incident such as resistance to heating, disinfectant, or the like (e.g., sporulation of hepatitis E virus living on food that is not completely cooked or the like), a modification state (methylation or the like) of a nucleic acid of a virus (e.g., hepatitis RNA virus, papilloma DNA virus, or coronavirus) that has infiltrated a host, and the like. The present disclosure is significant from the medical viewpoint in that cancers such as pancreatic cancer (e.g., early stage pancreatic cancer), liver cancer, gallbladder cancer, cholangiocarcinoma, gastric cancer, large intestinal cancer, bladder cancer, renal cancer, breast cancer, lung cancer, brain tumor, and skin cancer can be targeted. in one embodiment, the stage of cancer (e.g., pancreatic cancer) is analyzed by using the base sequence and/or modification information for a microRNA that has been obtained.

The present disclosure also can analyze responsiveness to an agent (e.g., anticancer agent, molecularly targeted drug, antibody drug, a biological formulation (e.g., nucleic acid or protein), an antibiotic, or the like) or treatment of a target organism. For example, drug resistance or the like can also be analyzed. The present disclosure can also be applied to, for example, analysis of responsiveness of an anticancer agent, selection of a suitable therapeutic agent, or antibiotic resistance or the like. The analysis of the present disclosure can also be used in analysis of prognosis or progress after surgery, radiation treatment or the like such as heavy particle beam (for example, Carbon/HIMAC) or X-ray treatment. It is understood that various drugs such as Lonsurf (TAS 102), gemcitabine, CDDP, 5-FU, cetuximab, a nucleic acid drug, and a histone demethylase inhibitor can be analyzed in the present disclosure, which can be utilized as basic information for a therapeutic strategy. If the agent is for example an anticancer agent, the present disclosure achieves establishment of a therapeutic strategy by testing the responsiveness as to whether a subject is resistant to the anticancer agent. Therefore, an agent for treating a subject and/or additional treatment for the subject can be selected based on responsiveness to treatment such as the agent in accordance with the present disclosure. When the responsiveness for a plurality of agents is studied, an agent for treating the condition can be indicated from among the plurality of agents in the present disclosure.

Analysis can be performed based on comparison of the base sequence and/or modification information (e.g., methylation) for a microRNA of the present disclosure in the subject before and after administration of an agent or the treatment.

In one embodiment of the present disclosure, a subject of analysis for a biological condition or a medical condition can be analyzed while further taking into consideration at least one piece of information selected from the group consisting of age, sex, race, familial information, medical history, treatment history, condition of smoking, condition of drinking, occupational information, information on living environment, disease marker information, nucleic acid information (including nucleic acid information on bacteria in the subject), metabolite information, protein information, enterobacterial information, epidermal bacterial information, and a combination thereof. Examples of nucleic acid information that can be utilized in the method of the present disclosure include genomic information, epigenomic information, transcriptome expression level information, RIP sequencing information, microRNA expression level information, and a combination thereof. RIP sequencing information that can be utilized individually can include RIP sequencing information on an agent-resistant pump P-glycoprotein, RIP sequencing information on a stool, RIP sequencing information on E. coli in a stool, or the like.

In the present disclosure, the condition of the subject can be analyzed further based on the base sequence and/or modification state of a microRNA in an agent or treatment-resistant strain, or a combination of the resistant strain and a cell strain from which the resistant strain is derived. Examples of such an agent or treatment include, but are not limited to, Lonsurf (TAS 102), gemcitabine, CDDP, 5-FU, cetuximab, a nucleic acid drug, a histone demethylase inhibitor, and a treatment using a heavy particle beam (e.g., Carbon/HIMAC) or an X-ray.

The types of microRNA subjected to analysis in the present disclosure can be increased or decreased in accordance with the objective of the analysis. For example, the base sequence and/or modification state of at least 5 types, at least 10 types, at least 20 types, at least 30 types, at least 50 types, at least 100 types, at least 200 types, at least 300 types, at least 500 types, at least 1000 types, at least 1500 types, or at least 2000 types of microRNAs can be analyzed. Alternatively, all available microRNAs can be targeted. In one embodiment, a plurality of pieces of modification information on microRNAs comprising the same sequence can be analyzed. In another embodiment, a condition of a subject can be analyzed further based on structural information of a microRNA.

In one embodiment, the method can comprise analyzing the condition of the subject further based on the base sequence and/or modification state of a microRNA in an organism with a knockdown of at least one of a methylase (e.g., Mett13, Mett114, or Wtap), a demethylase (e.g., FTC or AlkBH5), and methylation recognizing enzyme (e.g., family molecule with a YTH domain such as YTHDF1, YTHDF2, or YTHDF3) and/or recognition motif information on at least one of a methylase (e.g., Mett13, Mett114, or Wtap), a demethylase (e.g., FTC or AlkBH5), and methylation recognizing enzyme (e.g., family molecule with a YTH domain such as YTHDF1, YTHDF2, or YTHDF3).

One embodiment can perform calculation on a probability of a condition based on a plurality of pieces of modification information. Any statistical approach can be performed as the step of calculating, such as primary component analysis.

In one embodiment, the efficacy of an anticancer agent with accumulated clinical evidence (e.g., Lonsurf (TAS 102), gemcitabine, CDDP, 5-FU, cetuximab, a nucleic acid drug, or a histone demethylase inhibitor) on tumor tissue can be studied to establish a therapeutic strategy.

In one embodiment, a new mechanism of action of various agents can be elucidated to develop a middle molecule compound that can be applied in a further therapeutic strategy. For example, the compound can be utilized in drafting a strategy to overcome advanced refractory cancer.

In one embodiment, analysis of a microRNA with the approach of the present disclosure can further elucidate the mechanism of action. Specifically, a microRNA specific to an agent such as an anticancer agent can be analyzed using the method of the present disclosure, and a companion diagnostic drug can be designed using the same.

In one embodiment, companion diagnosis using an miRNA in peripheral blood obtained by minimally invasive liquid biopsy can be performed. For example, clinical information using an agent (e.g., Lonsurf (TAS 102), gemcitabine, CDDP, 5-FU, cetuximab, a nucleic acid drug, or a histone demethylase inhibitor) was collated with data for miRNAs in peripheral blood, and collated with mechanism analysis in a cell resistant to the agent, and miRNAs transcribed from chromatin in response to exposure to the agent and miRNAs secreted as exosomes in peripheral blood were able to be identified as 60 panels. The present disclosure provides a technology for identifying and analyzing such panels of microRNAs.

In one embodiment, the present disclosure can perform an analysis related to a cancer stem cell or Cancer Initiating Cell (CIC).

In one embodiment, the analysis of the present disclosure can also be applied when a modified RNA itself is a target molecule of a drug. Specifically, a novel agent can be screened by detecting whether an RNA is modified or unmodified using the analysis technology of the present disclosure. In particular, it was not known to apply a base sequence and/or modification state (e.g., methylation) of a microRNA in such screening for a novel agent in the past. The present disclosure can provide an agent with a new mechanism of action.

In another embodiment, the analysis of the present disclosure can also be applied when a modified RNA itself is a component molecule of a drug. For example, a novel agent can be screened by analyzing whether a target modified RNA or an external agent such as an enzyme responsible for the modification can be utilized as an agent by detecting whether an RNA is modified or unmodified using the analysis technology of the present disclosure.

In addition to the base sequence and/or modification state of a microRNA, the present disclosure can also combine and analyze other information on a nucleic acid such as information on base substitutions and/or modifications of a nucleic acid (DNA, RNA, or the like). Multi-omic analysis can be combined with a technology of multi-omic analysis of omics other than RNA modification (epitranscriptome) in Sijia Huang et al., Front Genet. 2017; 8:84, Yehudit Hasin et al., Genome Biol. 2017; 18:83 or the like.

Other information on nucleic acids can be analyzed by, for example, mass spectrometry or the like. For example, RIP-seq can be applied to RNAs, DIP-seq can be applied to

DNAs, and FDIP-seq can be performed with BrdU or the like for FDNAs to perform analysis.

In one embodiment, the analysis technology of the present disclosure can elucidate a new mechanism based on clinical evidence.

In still another embodiment, the present disclosure can study a drug development target. For example, a drug of a small or middle molecule compound can be developed, which targets the interaction between a complex of a plurality of molecules and a target. The technology for analyzing the base sequence and/or modification state of a microRNA of the present disclosure can be utilized when screening a library or screening a phenotype using an organoid or an individual animal.

In one embodiment, the present disclosure can be utilized in drug development that can handle tumor diversity. In addition to the base sequence and/or modification state of a microRNA of the present disclosure (e.g., methylation information of a microRNA), single molecule measurement of a modified DNA incorporating ChIP-seq or FTD, single cell analysis (C1) of lymphocytes or CAF (Cancer Associated Fibroblasts) of the stroma of tumor tissue, or the like can be combined and applied. When an agent is administered to a patient, the overall effect can be understood, including responses not only in cancer cells, but also in the host such as tumor stroma. If an inhibitor can be classified by utilizing information on the base sequence and/or modification state of a microRNA, an innovative drug that can differentiate the cancer cell space or stroma space can be developed.

In one embodiment, for a certain agent, the present disclosure can be applied to (1) expand indication for the agent to different indications, (2) demonstrate the superiority to other existing agents and move to 2^{nd} line therapy or earlier, (3) elucidate a new mechanism of action and investigate a possibility leading to a therapeutic drug, or the like.

In one embodiment, for example, expression information of an miRNA inside a serum exosome as a liquid biopsy of a patient such as a cancer patient can be prepared to analyze expression information of an miRNA inside a serum exosome of a patient after therapy of the subject of analysis or the base sequence and/or modification state of a microRNA of the present disclosure. For this reason, for colon cancer, expression information of an miRNA inside a serum exosome of an advanced colon cancer patient or base sequence and/or modification state thereof can be analyzed using, for example, a database for a total of 1000 cases (The Cancer Genome Atlas-Cancer Genome; TCCA).

Expression information of an miRNA inside a serum exosome of a colon cancer patient after therapy or base sequence and/or modification state thereof can also be analyzed.

In one embodiment, the present disclosure can provide a next generation RNA biomarker based on the base sequence and/or modification state of a microRNA based on the results of analysis. This can be clinically applied. For example, the present disclosure can find the tissue homeostasis from the base sequence and/or modification state of a microRNA and perform clinical applications using the same.

In analysis using information on an RNA of the present disclosure, it can be important that a target is a transcription factor, i.e., is an inducing agent that is a key to regulating (positively in many cases) expression of a target gene. In such a case, the number can be narrowed down by carefully selecting an independent transcription factor. For example, particularly noteworthy is that it was found “c-myc” having action as a cancer gene can be let go early in cancer diagnosis if the method of the present disclosure is used. It is understood that limited independence of a transcription factor is lost in the presence of a cancer gene, and various actions are manifested in a cell context dependent manner, so that the minimum number cannot be found clearly. It was found that in such a case, “c-myc” would be noise since c-myc acts on many sideway actions.

In the present disclosure, an miRNA (also referred to as “microR”, “microRNA”, or “miR”) is used. Such a case is characterized in having many-to-many relationship. Specifically, one of the important points is that a single microR acts on many, and shares a common target between microRNAs as different molecules. It is not surprising that, given that there is an important set inducing a certain event, this is not a single molecule in such a regulatory system with “many-to-many relationship”. Rather, this being a limited set, and being expressable with weightings that can express the hierarchy within the set are features of analysis provided by the present disclosure.

In one embodiment, cancer diagnosis with the base sequence and/or modification state of a microRNA is envisioned. This is not limited thereto. Additionally, agent resistance (not only anticancer agent, but also molecularly targeted drug, antibody drug, nucleic acid, and other biological formulations, and more broadly a microorganism-derived antibiotic or the like), classification of a population of species, inflammatory bowel disease, E. coli, food classification (production region, age, taste, quality, expiration date, sense of taste) and the like are also envisioned. The base sequence and/or modification state of a microRNA can be used for selecting koji yeast.

In one embodiment, it is known for example that other agents such as 5-FU and CDDP have significantly different IC50 distributions, where 5-FU is very effective when effective, but almost completely ineffective when ineffective. This can be found using the epitranscriptome. For example, for microRNAs, it is known that classification lines can be drawn more precisely by largely differentiating with primary component analysis (PCA) from studying the epitranscriptome rather than studying the expression.

RNA methylation is known to be associated with Circadian rhythm (Sanchez et al., Nature. 2010 Nov. 4; 468 (7320): 112-6, Jean-Michel et al., Cell Vol. 155, Issue 4, pp. 793-806 7 November 2013, and the like). In one embodiment, the base sequence and/or modification state of a microRNA can be used to analyze sleep activity related to time difference (jet lag, etc.) For example, determination of the possibility of an impact of time difference on sleep activity of a subject, personnel and medical management associated therewith, management of a pilot or flight attendant, stratification of whether dosing of melatonin is recommended, or the like can be performed based on such analysis. In one embodiment, the base sequence and/or modification state of a microRNA can be used to analyze jet lag from space flight.

In one embodiment, the base sequence and/or modification state of a microRNA can be used to analyze whether sleep of a subject is sufficient. Although latent sleep deprivation is an issue, the subject is not self-aware in many cases. In this regard, the base sequence and/or modification state of a microRNA can be used for the correction thereof. In one embodiment, this is matched with sleep habit therapy. In one embodiment, the base sequence and/or modification state of a microRNA can be used to manage the health of long distance bus drivers. In one embodiment, the base sequence and/or modification state of a microRNA can be used for welfare management. In one embodiment, the base sequence and/or modification state of a microRNA can be used to manage the health of night shift workers (steel manufacturing plant, nuclear power plant, hospital workers, medical practitioners, security guards, building management company's employee, etc.)

In one embodiment, the age of a subject can be analyzed using the base sequence and/or modification state of a microRNA in a blood sample for use in crime investigation.

In one embodiment, the base sequence and/or modification state of a microRNA can be used to analyze the presence/absence of doping.

(Biomarker Screening)

In one embodiment, a new biomarker can be searched using the obtained base sequence and/or modification state of a microRNA. In one embodiment, the base sequence and/or modification state of a microRNA obtained in a subject in a certain condition can be compared to the base sequence and/or modification state of a microRNA obtained in a subject who is not in such a condition, and an RNA or group of RNAs observed to have a difference (e.g., statistically significant difference) in the base sequence and/or modification state of a microRNA can be used as a biomarker for predicting the condition.

In one embodiment, the base sequence and/or modification state of a microRNA obtained in a subject administered with a drug and/or treatment can be compared to the base sequence and/or modification state of a microRNA obtained in a subject who is not administered with such a drug and/or treatment, and an RNA or group of RNAs observed to have a difference (e.g., statistically significant difference) in the base sequence and/or modification state of a microRNA can be used as a biomarker for predicting the responsiveness and/or resistance to the drug and/or treatment.

(Resistant Strain)

In one embodiment, the base sequence and/or modification state of a microRNA obtained in a resistant strain with resistance to a drug and/or treatment can be compared to the base sequence and/or modification state of a microRNA obtained in a wild-type strain from which the resistant strain originated, and an RNA or group of RNAs observed to have a difference (e.g., statistically significant difference) in the base sequence and/or modification state of a microRNA can be used as a biomarker for predicting the responsiveness and/or resistance to the drug and/or treatment.

Such a resistant strain can be prepared, for example, by maintenance culture of a wild-type strain in the presence of a drug and/or treatment. In one aspect, the present disclosure provides a method of preparing such a resistant strain. In one aspect, a strain resistant to a drug and/or treatment can be evaluated as to whether the strain is a resistant strain based on IC₅₀ with respect to the drug and/or treatment. In one aspect, the present disclosure provides a resistant strain with resistance to each of trifluridine (FTD), 5-fluorouracil (5-FU), gemcitabine, cisplatin, Carbon/HIMAC (heavy particle beam), and X ray.

(Drug Screening)

In one embodiment, a new drug can be evaluated using the obtained base sequence and/or modification state of a microRNA. In one embodiment, the base sequence and/or modification state of a microRNA obtained in a subject treated with a certain drug can be compared to the base sequence and/or modification state of a microRNA obtained in a subject treated with another drug for classification of drugs based on an RNA changed by treatment with each drug.

(Species Classification)

In one embodiment, organism species can be classified by using the obtained base sequence and/or modification state of a microRNA. In one embodiment, a subject (e.g., mammal such as a human, food, or the like) from which a microorganism (e.g., E. coli) was obtained can be analyzed based on a result of classifying the microorganism.

(Food)

In one embodiment, quality of food can be analyzed using the obtained base sequence and/or modification state of a microRNA. Examples of quality of food include, but are not limited to, production region, age, time since processing, freshness, denaturation after processing, quality of taste, condition of active oxygen, microorganism contamination (E. coli, Salmonella, Clostridium botulinum, virus, parasite, and the like), fermentation condition (including condition of microorganisms associated with fermentation), chemical factors (e.g., pesticides, additives, and the like), physical factors (e.g., foreign objects, radiation, and the like), condition of fatty acid, degree of maturation, and the like. In one embodiment, quality of food can be analyzed using the base sequence and/or modification state of a microRNA obtained for controlling quality of food by a public institution such as a governing body. In one embodiment, quality of food can be analyzed by using the base sequence and/or modification state of a microRNA obtained to provide an indicator for a consumer to determine the quality of a product (objectively express quality which was expressed by taste or odor).

Unlike DNAs, RNAs, and proteins, RNA modifications (e.g., methylation) provide a new development, when viewed from a different viewpoint, in the present disclosure. For example, DNAs and RNAs lose information on contiguous base sequences upon degradation (become short and fragmented). Methylation is expressed as a methylation ratio as an indicator expressing the quality thereof, as long as there is a target site. Thus, this is unique in that “how the original factors diminish and remain during chronological changes” can be monitored. Proteins are not only in the middle thereof, but a target is not determined in the present case, such that proteins have limitations as a tracking tool or a tracer. Therefore, modifications attain a particularly significant effect unlike DNAs, RNAs, and proteins.

(Utilization of Additional Information)

In one embodiment, a condition of a subject can be analyzed by using the base sequence and/or modification state of a microRNA obtained from the subject as well as other information, such as the base sequence and/or modification state of a microRNA obtained from the subject at another time (e.g., before and after treatment), information related to the subject, information on a motif of a protein associated with a modification, information related to the base sequence and/or modification state of a microRNA obtained from another subject, information related to a complex of a substance binding to an RNA (protein, lipid, or the like) and the RNA (optionally, an additional condition associated with an RNA modification state), and the like.

Examples of information related to a subject that can be additionally used include the subject's age, sex, race, familial information, medical history, treatment history, condition of smoking, condition of drinking, occupational information, information on living environment, disease marker information, nucleic acid information (including nucleic acid information of bacteria in the subject), metabolite information, protein information, enterobacterial information, epidermal bacterial information, and the like. Examples of nucleic acid information include genomic information, genomic modification information, transcriptome information (including information on the expression level and sequence), RIP sequencing information, and microRNA information (including information on the expression level and sequence). Examples of RIP sequencing information that can be used individually include RIP sequencing information on an agent-resistant pump P-glycoprotein, RIP sequencing information on a stool, RIP sequencing information on E. coli in a stool, and the like.

Examples of motif information of a protein associated with a modification that can be additionally used include information on a recognition motif of an enzyme adding a modification, information on a recognition motif of an enzyme that removes a modification, and information on a recognition motif of a protein that binds to a modification. Specific examples thereof include motif information on methylase (e.g., Mett13, Mett114, and Wtap), demethylase (e.g., FTO and AlkBH5), and methylation recognizing enzyme (e.g., family molecules with a YTH domain such as YTHDF1, YTHDF2, or YTHDF3).

Examples of information related to RNA modifications obtained from another subject that can be additionally used include, but are not limited to, RNA modification information in a subject having a certain condition, RNA modification information in an organism genetically engineered for expression of a protein associated with a modification, RNA modification information in a resistant strain having resistance to a drug and/or treatment, RNA modification information in a subject administered with a drug and/or treatment, and information related to a complex of a substance binding to an RNA (protein, lipid, or the like) and the RNA (optionally a condition associated with an RNA modification state).

In the present disclosure, the condition of a subject can be analyzed further based on the base sequence and/or modification state of a microRNA in an agent or treatment resistant strain or a combination of the resistant strain and a cell strain from which the resistant strain is derived. Examples of such an agent or treatment include, but are not limited to, Lonsurf (TAS 102), gemcitabine, CDDP, 5-EU, cetuximab, a nucleic acid drug, a histone demethylase inhibitor, and a treatment using a heavy particle beam (e.g., Carbon/HIMAC) or an X-ray.

(Subject Condition Analysis Method)

In one embodiment, the present disclosure provides a method of analyzing a condition of a subject, from whom a microRNA has been obtained by referring to accumulated data for a combination of the base sequence and/or modification state of the microRNA and a pattern of a tunneling channel, to analyze the base sequence and/or modification state of the microRNA based on the detected pattern of the tunneling current. The base sequence and/or modification state can be obtained by measuring a sample derived from a subject. In one embodiment, analysis is an onsite analysis for taking measurement in a short period of time (e.g., 1 day or less, 10 hours or less, 5 hours or less, 2 hours or less, 1 hour or less, 30 minutes or less, 15 minutes or less, or the like) after obtaining a sample. In one embodiment, a result of onsite analysis is outputted in a short period of time after obtaining a sample (e.g., 1 day or less, 10 hours or less, 5 hours or less, 2 hours or less, 1 hour or less, 30 minutes or less, 15 minutes or less, or the like). In one embodiment, after a sample is obtained, the sample is delivered to a location of a measurement instrument and/or analyzer, where analysis is performed. A sample can be obtained by the subjects themselves. In one embodiment, an obtained sample is frozen and delivered. A result of analysis can be sent to the sender, or made available through accessing an Internet site.

If the method of the present disclosure is practiced for example in a medical institution such as a hospital, a sample (e.g., blood, extracted organ, stool, or the like) is obtained from a subject (e.g., a patient, a subject at risk of a disease or the like) , and the sample is treated to purify a microRNA of interest to identify the base sequence and/or modification state of the microRNA of interest. A condition (e.g., possibility of development or recurrence or cancer, possibility of acquiring resistance to a specific drug therapy, or the like) of a subject can be analyzed based on the base sequence and/or modification state of a microRNA identified in this manner. The base sequence and/or modification state of a microRNA, once obtained, can be used in analysis of a condition of another subject, used in analysis of a condition at another time in the same subject, or accumulated in a database.

If, for example, the method of the present disclosure is practiced at a research institution of a pharmaceutical company, etc., or a medical facility, or a company providing a service of conducting a clinical test or the like that has been commissioned for service therefrom, a sample obtained from a subject (e.g., tissue or organ of an experimental animal, clinical sample, cultured cell, or the like) is treated to purify a microRNA of interest to identify the base sequence and/or modification state of the microRNA of interest. The base sequence and/or modification state of a microRNA identified in this manner can be accumulated while being associated with a condition of the same subject found by another analysis (e.g., condition of cancer, condition of having acquired drug resistance, condition of a drug attaining a therapeutic effect, or the like). A drug that can be suitably applied to a condition of a subject (patient, a subject at risk of a disease, or the like) can be determined based on the base sequence and/or modification state of a microRNA obtained in this manner.

(Program)

In one aspect, the present disclosure provides a program configured to implement, on a computer, a method comprising: inputting a result of analysis of a microRNA by a mass spectrometer; inputting a pattern of a tunneling current obtained by tunneling current measurement on the microRNA; and determining a modification state of the microRNA by associating the result of analysis by mass spectrometry with the pattern of the tunneling current.

In one aspect, the present disclosure provides a program configured to implement, on a computer, a method of analyzing a subject, the method comprising: obtaining a pattern of a tunneling current by tunneling current measurement on a microRNA of the subject; referring to a database comprising a combination of a modification state of the microRNA and the pattern of a tunneling current already obtained by tunneling current measurement to analyze the modification state of the microRNA of the subject based on the obtained pattern of a tunneling current; and analyzing a condition of the subject based on the modification state. In one embodiment, the method further comprises showing a condition of the subject based on the modification state of the microRNA of the subject.

In one aspect, the present disclosure provides a program that implements, on a computer, a method of analyzing a condition of a subject based on a base sequence and/or modification state of a microRNA, and a recording medium for storing the program. The method executed by the program comprises: (a) comparing a base sequence and/or modification state of at least one type of microRNA in a subject with a reference base sequence and/or modification state of the microRNA; and (b) determining the condition of the subject based on an output result of the comparison. In one embodiment, reference modification information comprises the base sequence and/or modification state of a microRNA in a subject that is different from the subject. In one embodiment, reference modification information comprises a base sequence and/or modification state of the microRNA in the subject obtained at another time different from the time when the base sequence and/or modification state obtained.

(System)

In one aspect, the present disclosure provides a system for associating a base sequence and/or modification state of a microRNA with a pattern of a tunneling current obtained by tunneling current measurement. The system comprises: a mass spectrometer; a tunneling current meter; and an analysis/determination unit for analyzing and determining a base sequence and/or modification state of a microRNA of interest by associating results of measuring the microRNA of interest by the mass spectrometer and tunneling current measurement.

In one aspect, the present disclosure provides a system for analyzing a condition of a subject based on a base sequence and/or modification information of a microRNA. The system comprises: a tunneling current meter; and an analysis/determination unit for referring to accumulated data on a combination of base sequence and/or modification information for a microRNA and a pattern of a tunneling current obtained by tunneling current measurement, to analyze and determine the base sequence and/or modification state of a microRNA of a subject from whom the microRNA has been obtained based on the pattern of a tunneling current obtained by tunneling current measurement on a microRNA. In one embodiment, the system further comprises a condition analysis/determination unit for analyzing and determining a condition of the subject based on the analyzed and determined base sequence and/or modification state.

A measurement unit can have any configuration, as long as the unit has a function and arrangement for providing the base sequence and/or modification state of a microRNA.

The unit can be provided as the same or different structure as the calculation unit or analysis unit. In one embodiment, a measurement unit comprises a tunneling current meter. In one embodiment, the measurement unit comprises a mass spectrometer (e.g., MALDI-MS).

A calculation unit identifies the base sequence and/or modification state of a microRNA based on measurement data.

An analysis unit analyzes a condition of a subject based on obtained microRNA information. In one embodiment, analysis can be performed by referencing the additional information described above.

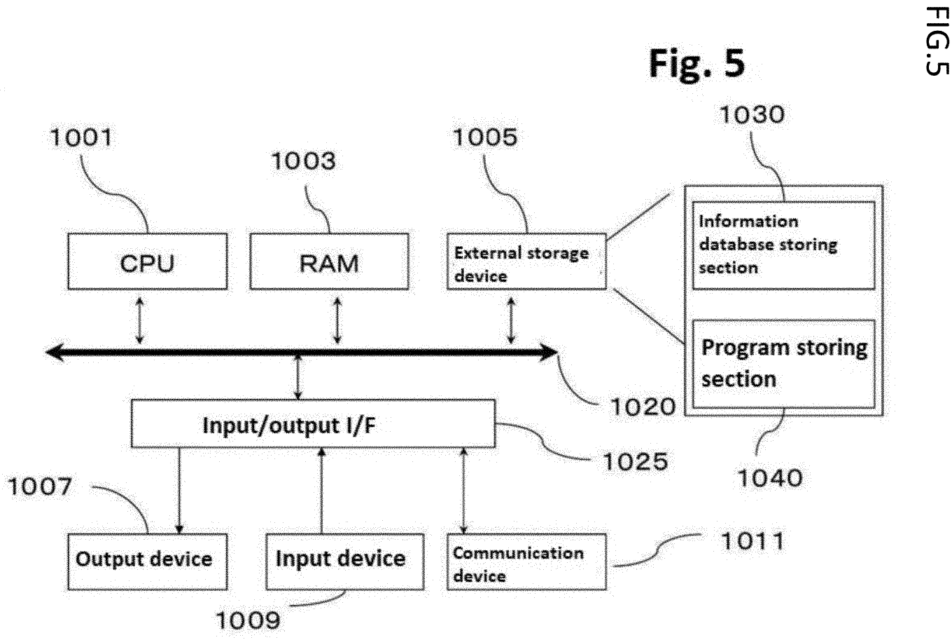

The configuration of the system of the present disclosure is described while referring to the functional block diagram in FIG. 5. While this figure shows a case materializing the present disclosure in a single system, it is understood that a case materializing the invention with a plurality of systems is also encompassed within the scope of the present disclosure. A method materialized with this system can be described as a program. Such a program can be recorded on a recording medium and materialized as a method.

The system 1000 of the present disclosure is constituted by connecting a RAM 1003, a ROM, SSD, or HDD or a magnetic disk, an external storage device 1005 such as a flash memory, such as a USB memory, and an input/output interface (I/F) 1025 to a CPU 1001 built into a computer system via a system bus 1020. An input device 1009 such as a keyboard or a mouse, an output device 1007 such as a display, and a communication device 1011 such as a modem are each connected to the input/output I/F 1025. The external storage device 1005 comprises an information database storing section 1030 and a program storing section 1040, which are both constant storage areas secured within the external storage apparatus 1005.

In such a hardware configuration, various instructions (commands) are inputted via the input device 1009 or commands are received via the communication I/F, communication device 1011, or the like to call, expand, and execute a software program installed on the storage device 1005 on the RAM 1003 by the CPU 1001 to achieve the function of the present disclosure in cooperation with an OS (operating system). Of course, the method of the present disclosure can be implemented with a mechanism other than such a cooperating setup.

In the implementation of the method of the present disclosure, microRNA data, when obtained by measuring (e.g., by mass spectrometry and/or tunneling current measurement) a microRNA sample, or information equivalent thereto (e.g., data obtained by simulation) can be inputted via the input device 1009, inputted via the communication I/F, communication device 1011, or the like, or stored in the database storing section 1030. The step of obtaining microRNA data by measuring (e.g., by mass spectrometry and/or tunneling current measurement) the microRNA sample and analyzing the microRNA data can be executed with a program stored in the program storing section 1040, or a software program installed in the external storage device 1005 by inputting various instructions (commands) via the input device 1009 or by receiving commands via the communication I/F, communication device 1011, or the like.

As the software for performing such analysis, any software known in the art can be used. Analyzed data can be outputted through the output device 1007 or stored in the external storage device 1005 such as the information database storing section 1030.

The data or calculation result or information obtained via the communication device 1011 or the like is written and updated immediately in the database storing section 1030. Information attributed to measurement data subjected to accumulation (measurement condition, origin of sample, etc.) can be managed with an ID defined in each master table by managing information such as each of the sequences in each input sequence set and each RNA information ID of a reference database in each master table.

The above calculation result can be associated with various information such as other nucleic acid information obtained from the same sample or known information such as biological information, and can be stored in the database storing section 1030. Such association can be performed directly to data available through a network (Internet, Intranet, or the like) or as a link to the network.

A computer program stored in the program storing section 1040 is a constituent of a computer as the above processing system, e.g., a system for performing data provision, extraction of features of tunneling current measurement data, identification of the base sequence and/or modification state, comparison with reference data, classification, clustering, or other processes. Each of these functions is an independent computer program, a module thereof, or a routine, which is executed by the CPU 1001 to use a computer as each system or device.

(Reagent)

In one aspect, the present disclosure provides a composition for purifying a microRNA to determine a condition of a subject based on the microRNA, comprising an agent (e.g., reagent, capturing agent, etc.) for capturing at least one type of microRNA in the subject. In one embodiment, the capturing agent comprises a nucleic acid that is at least partially complementary to a microRNA of interest. In one embodiment, a capturing agent comprises an agent for capturing a modified RNA (e.g., modification specific antibody or the like). In one embodiment, a capturing agent comprises a molecule specific to a modified RNA of interest. In one embodiment, a capturing means comprises a portion for purification (e.g., a carrier that can be magnetic or one side of a pair that can bind to each other (e.g., biotin and streptavidin)). In one embodiment, a capturing means comprises a linker linked to a portion for purification.

(Kit)

In one embodiment, the present disclosure provides a kit for determining a condition of a subject based on a microRNA, comprising at least one of a composition for purifying a microRNA of interest and a device for obtaining a sample from the subject, and descriptions for using the kit. In one embodiment, a kit comprises means for purifying an RNA from a sample.

In one embodiment, a kit comprises a device for obtaining a sample from a subject. In one embodiment, a kit comprising a device for obtaining a sample from a subject comprises descriptions describing where a sample is to be sent. In one embodiment, a kit comprises means for cryopreserving a harvested sample. In one embodiment, a kit comprises a device for obtaining, from a subject, blood, epidermis of the mucous membrane (e.g., in the oral cavity, nasal cavity, ear cavity, vagina, or the like), epidermis of the skin, biological secretion (e.g., saliva, nasal mucus, sweat, tear, urine, bile, or the like), stool, or epidermal microorganism.

(General Technology)

The molecular biological approaches, biochemical approaches, and microbiological approaches used herein are well known or conventional in the art, which are described for example in Current Protocols in Molecular Biology (http://onlinelibrary.wiley.com/book/10.1002/0471142727) and Molecular Cloning: A Laboratory Manual (Fourth Edition) (http://www.molecularcloning.com). The relevant portions (can be the entire document) thereof are incorporated herein by reference.

As used herein, “or” is used when “at least one” of the elements listed in the sentence can be used. When explicitly described herein as “within a range” of “two values”, the two values themselves are included in the range.

Reference literatures such as scientific literatures, patents, and patent applications cited herein are incorporated herein by reference to the same extent that the entirety of each document is specifically described.

As described above, the present disclosure has been described while showing preferred embodiments to facilitate understanding. While the present disclosure is described hereinafter based on Examples, the above descriptions and the following Examples are not provided to limit the present disclosure, but for the sole purpose of exemplification. Thus, the scope of the invention is not limited to the embodiments and Examples specifically described herein and is limited only by the scope of claims.

### EXAMPLES

For reagents, the specific products described in the

Examples were used. However, an equivalent product from another manufacturer (Sigma-Aldrich, Wako Pure Chemical, Nacalai Tesque, R & D Systems, USCN Life Science INC, or the like) may be alternatively used.

### (Example 1) Preparation of Electrode Pair

An electrode pair was prepared through nanofabricated mechanically-controllable break junctions (MCBJ) (see Tsutsui, M., Shoji, K., Taniguchi, N., Kawai, T., Formation and self-breaking mechanism of stable atom-sized junctions. Nano Lett. 8, 345-349 (2007)). The preparation method of an electrode pair is briefly described hereinafter.

Nano-scale gold junction was patterned on a flexible metal substrate (phosphor bronze substrate) coated with polyimide (Industrial Summit Technology, catalog number: Pyre-M1) by standard electron-beam lithography and lift-off technology using an electron-beam lithography system (JEOL, catalog number JSM6500F).

Next, the polyimide under the junction was removed by etching based on reactive ion etching by using a reactive ion etching system (Samco, catalog number: 10NR). A nano-scale gold bridge with a structure bent at three points was prepared by bending the metal substrate. The substrate was bent in this manner using a piezo actuator (CEDRAT, catalog: APA150M). The distance between the electrodes of the electrode pair can be controlled at a resolution of picometer or less by precisely bending the substrate.

The bridge was then pulled to break a part of the bridge to form an electrode pair (gold electrodes). Specifically, the bridge was pulled and broken by applying 0.1 V of DC bias voltage (Vb) to the bridge using 10 kΩ of resistance in series under a programmed junction pulling rate through the resistance feedback method (see M. Tsutsui, K. Shoji, M. Taniguchi, T. Kawai, Nano Lett. 8, 345 (2008), and M. Tsutsui, M. Taniguchi, T. Kawai, Appl. Phys. Lett. 93, 163115 (2008)) by using a data acquisition board (National Instruments, catalog number: NI PCIe-6321). The bridge was pulled further, so that the size of the gap generated by the breakage (distance between electrodes) was set to the length of the nucleotide molecule of interest (about 1 nm).

The electrode pair prepared in this manner was observed under a microscope.

### (Example 2) Measurement of Tunneling Current on Synthetic MicroRNA

The following microRNAs were synthesized.

miR-200c-5p
(SEQ ID NO: 1)
5′-CGUCUUACCCAGCAGUGUUUGG-3′
miR-200c-5p
(SEQ ID NO: 1)
5′-CGUCUUACCCAGCAGUGUUUGG-3′ (#7, mA; #13, mC)

These synthetic microRNAs were each added to Milli-Q so that the final concentration would be 0.10 μM to prepare a solution for measurement.

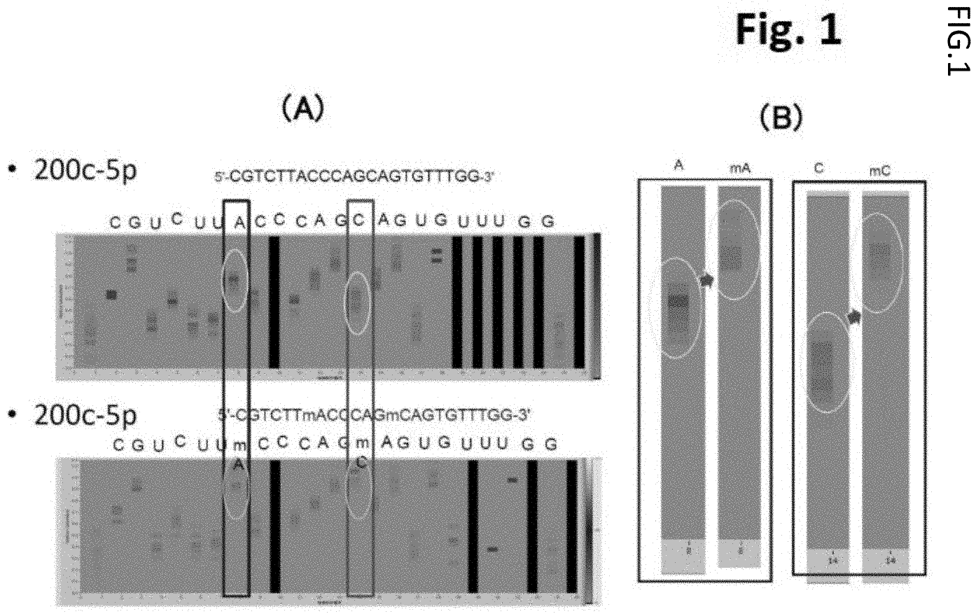

The electrode pair was immersed in the solution for measurement, and a voltage of 0.4 V was applied between the electrode pair to measure a tunneling current that was generated between the electrode pair. At this time, the synthetic microRNA that was present between the electrodes was in Brownian motion (temperature of the solution for measurement was about 25° C.). The tunneling current was measured using a logarithmic amplifier (manufactured at Daiwa GiKen Co. Ltd. in accordance with the design described in Rev. Sci. Instrum. 68(10), 3816) and PXI 4071 digital multimeter (National Instruments) at 10 kHz under a DC bias voltage of 0.4 V. The results are shown in FIG. 1.

### (Example 3) Mass Spectrometry on Synthetic MicroRNA

The synthetic microRNAs prepared in Examples 2 are subjected to mass spectrometry.

3-HPA (3-hydroxypicolinic acid) is added to a solution of acetonitrile:aqueous 0.1% TFA solution =1:1 so that the concentration would be 10 mg/mL. 1 μL of mixture prepared by mixing this solution with an aqueous 10 mg/mL DHC (diammonium citrate) solution at a ratio of 1:1 is applied to a target plate (Target Plate MTP Anchor Chip 384 (600 micrometer), Bruker Daltonics) as a MALDI matrix (coating agent) and dried. At the same position, 1 μL of purified aqueous RNA solution is coated thereon and dried. After confirming that it is fully dry, mass spectrometry is performed with a MALDI mass spectrometer (ultrafleXtreme-TOF/TOF mass spectrometer, Bruker Daltonics).

The MALDI system is set as follows.

positive-mode (positive ion detection mode)
reflector-mode (reflector mode)
Laser Power Max (maximum settable output)

(Analysis of Results of Measurement on Mass Spectrometer)

The measurement results obtained by MALDI are analyzed as follows.

A list of expected masses is created based on sequence information for microRNAs obtained from miRBase (Release 21) (http://www.mirbase.org), and sequences and modifications are manually identified by comparison with a mass spectrogram obtained by measurement.

### (Example 4) Measurement on MicroRNA Obtained from Sample

A DNA complementary to 200c-5p with the following sequence (capturing 200c-5p) was synthesized.

Capturing 200c-5p
(SEQ ID NO: 2)
CCAAACACTGCTGGGTAAGACG

(Streptavidin Binding Beads)

Biotin was introduced into a phosphoric acid moiety at the 5′ end of capturing 200c-5p. Magnetic beads (Dynabeads M270 Streptavidin, Thermo Fisher Scientific) with streptavidin covalently bound to the surface were mixed with the biotinylated capturing oligo DNAs described above to generate an avidin-biotin bond and immobilize the captured oligo DNA on the magnetic beads.

(Purification Protocol)

1. Saline and the biotinylated capturing 200c-5p were added to the purified RNA to prepare a phosphate buffer with a final concentration of 10 mM (pH 7.0) and 50 mM KCl.
2. The mixture was placed in a PCR system, denatured for 1 minute at 90° C., gradually cooled to 45° C., and annealed.
3. Avidin magnetic beads (Dynabeads M-280 Streptavidin) were added.
4. The unadsorbed fraction (supernatant), on the magnet stand, was discarded.
5. The beads were washed three times on the magnet stand using 10 mM phosphate buffer (pH 7.0) and 50 mM KCl.
6. The beads were washed three times on the magnet stand using 10 mM ammonium acetate (pH 7.0), which was then eluted using RNase free water.
7. When the concentration was low, the supernatant was lyophilized.

(Sample Preparation)

200c-5p was concentrated from a sample by using the streptavidin binding beads bound to the capturing 200c-5p described above. The concentrated 200c-5p was added to

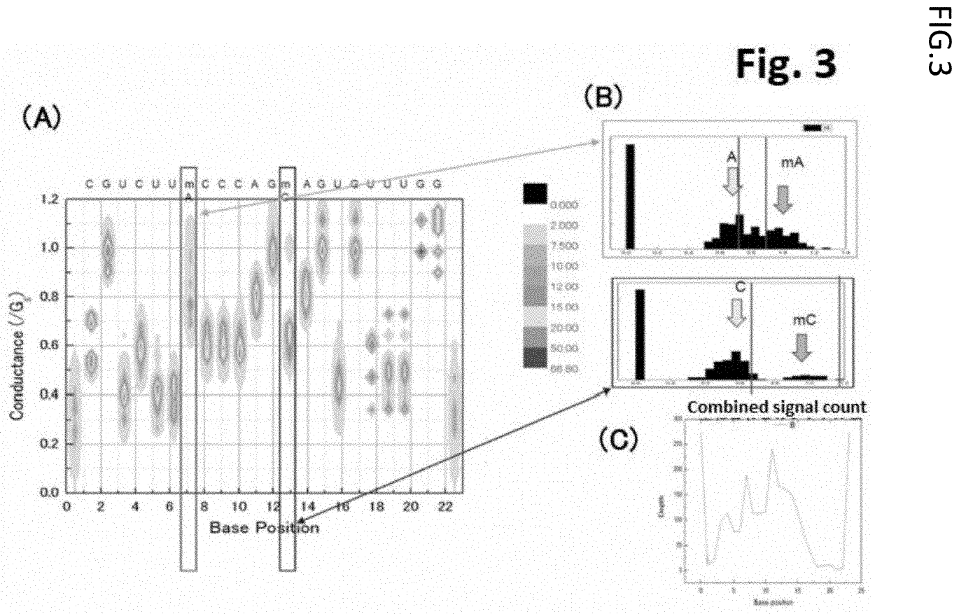

Milli-Q so that the final concentration would be 0.10 μM to prepare a solution for measurement. A tunneling current was measured in the same manner as Example 2. The results from comparison with the synthetic microRNAs prepared in Example 2 are shown in FIGS. 2 and 3.

### (Example 5) Identification of MicroRNAs with Different Modified Positions on a Structure

A tunneling current is measured for microRNAs with different modified positions on a structure to analyze modifications.

### (Example 6) Search for Biomarkers by Combining Tunneling Current Measurement and Mass Spectrometry

Samples are prepared from serum from a cancer patient and serum from a healthy individual, and a tunneling current is measured to analyze a modification of a microRNA. A microRNA with significantly more modifications in a cancer patient than a healthy individual is searched.

### (Example 7) Determination of Disease Based on Results of Measuring Tunneling Current

DNAs complementary to let7a-5p or miR17-5p with the following sequences (capturing let7a-5p and capturing miR17-5p) were synthesized.

Capturing 17-5p
(SEQ ID NO: 3)
CTACCTGCACTGTAAGCACTTTG
Capturing let7a-5p
(SEQ ID NO: 4)
AACTATACAACCTACTACCTCA

Bodily fluid was collected from human pancreatic cancer patients (stage I to stage IV pancreatic cancer) and healthy individuals. let7a-5p and miR17-5p were concentrated with streptavidin binding beads in the same manner as Example 4 by using capturing let7a-5p and capturing miR17-5p for these bodily fluid samples.

The concentrated let7a-5p and miR17-5p were added to Milli-Q so that the final concentration would be 0.10 μM to prepare a solution for measurement. A tunneling current was measured in the same manner as Example 2. Chronological signal data for conductance values was obtained and assembled for each miRNA sequence to create a histogram of conductance values. The relative conductance value for adenine and conductance value for methylated adenine in monomer data are 0.7 and 0.8, respectively. Meanwhile, the conductance values for adenine and methylated adenine in a nucleic acid sequence can be in the range of 0.60 to 0.8 and 0.75 to 0.90, respectively, depending on the peak position and shape of the histogram. Thus, each signal corresponding to a position of adenine was determined as either adenine or methylated adenine by using a probability density from a Gaussian function or the like as an indicator. The number of adenine and the number of methylated adenine were counted based on such a determination result, and the methylation ratio (amount) was computed as methylated adenine count/(adenine count+methylated adenine count).

The results are shown in FIG. 6. A difference was found in the methylation ratios at specific positions in let7a-5p and miR17-5p (let7a-5p: adenine at positions 10, 17, and 19, miR17-5p: adenine at position 11) between a pancreatic cancer patient and healthy individual. Specifically, the methylation ratio for adenine at each position in let7a-5p for each patient (pancreatic cancer patient, healthy individual) were (0%, 0%) for adenine at position 3, (0%, 0%) for adenine at position 7, (5.2%, 0%) for adenine at position 10, (13.0%, 0%) for adenine at position 17, and (5.2%, 0%) for adenine at position 19. A pancreatic cancer patient can be indicated based on such methylation ratios of a microRNA or change (increase) in the methylation ratio at a specific position.

Similarly, a disease is determined by measuring a tunneling current for a sample obtained from a subject with an inflammatory bowel disease, Crohn's disease, diabetes, and psychiatric disease, and by analyzing a modification of a microRNA.

A sample is prepared from a serum from a cancer patient and serum from a healthy individual, a tunneling current is measured, and a modification of a microRNA is analyzed to determine a disease.

### (Example 8) Quantitative Analysis by Tunneling Current Measurement

Quantitative analysis of a microRNA was performed by tunneling current measurement. Colon cancer cell strain (DLD1) and 5-fluorouracil (5-FU) or trifluridine (FTD) resistant strains were used as samples.

Resistant strains were prepared in the following manner.

Cancer cell strain DLD-1 obtained from a cell bank was maintained in a culture for 6 months or longer in the presence of trifluridine or 5-fluorouracil (Aldrich-Sigma) (about 10 mg/mL). The maintained culture was passaged about twice a week to maintain 60 to 80% confluence at 37° C. in a

DMEM medium supplemented with 10% serum on a plastic dish. Measurement for the IC₅₀ with respect to trifluridine or 5-fluorouracil for the cultured cells resulted in IC₅₀=300 μmol/L, which confirmed the establishment of a resistant strain.

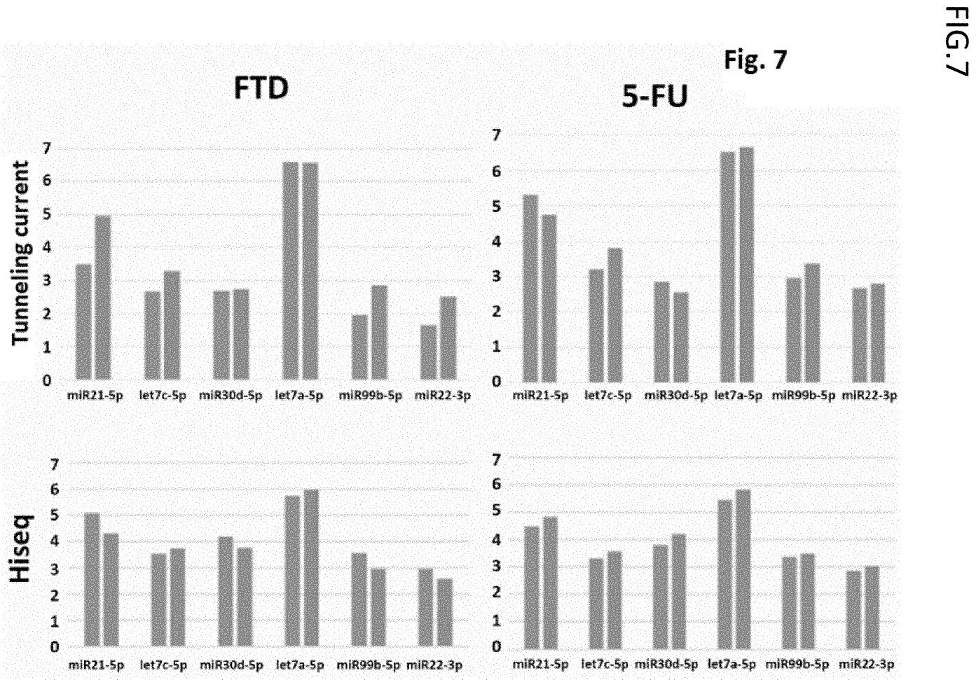

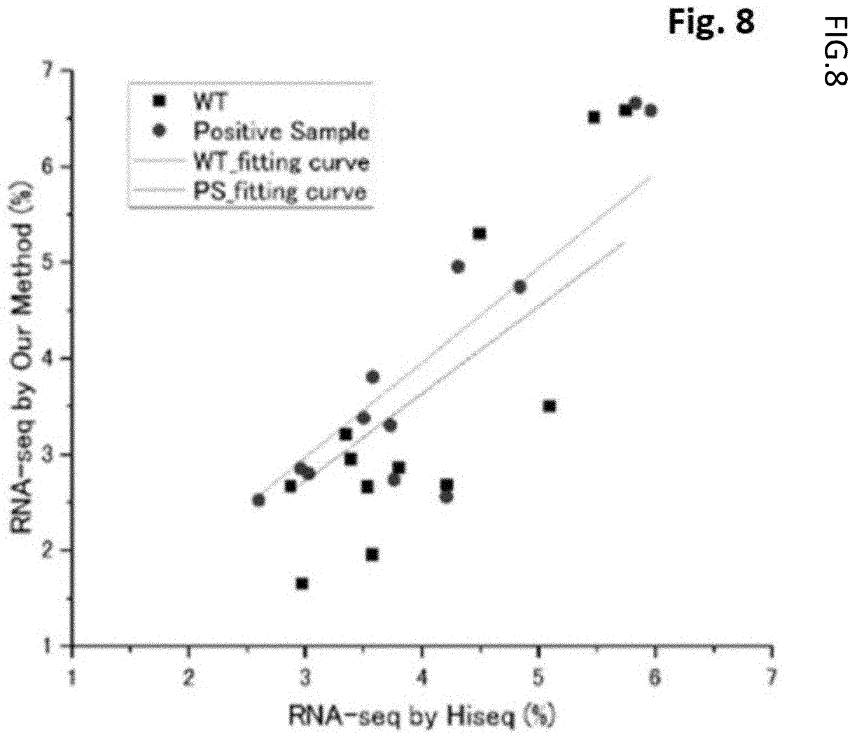

Total RNA was extracted from each cell strain by using TRIzol (invitrogen) in accordance with the user manual. RNA comprising m6A was concentrated from the total RNA by immunoprecipitation using an anti-m6A antibody (Santa Cruz Biotechnology). The anti-m6A antibody concentrated RNA was subjected to tunneling current measurement similar to that in Example 2 and measurement using Hiseq 2000 (Illumina, Calif.). The results are shown in FIGS. 7 and 8. The quantitative analysis results exhibited a high correlation between measurement by a commonly used sequencer and the tunneling current measurement of the present disclosure, suggesting that the tunneling current measurement of the present disclosure allows convenient and highly reliable quantitative analysis.

(Note)

The present application claims priority to Japanese Patent Application No. 2019-85805 filed on Apr. 26, 2019. The entire content thereof is incorporated herein by reference.

### INDUSTRIAL APPLICABILITY

The present disclosure provides a method of identifying a base sequence and/or modification state of a microRNA by using a tunneling current, and a system and program for use in this method. A condition of a subject (e.g., medical condition) can be analyzed by identifying the base sequence and/or modification state of a microRNA by using a tunneling current.

[Sequence Listing Free Text]
*SEQ ID NO: 1: naturally-occurring human 200c-5p
sequence
CGUCUUACCCAGCAGUGUUUGG
*SEQ ID NO: 2: capturing 200c-5p
CCAAACACTGCTGGGTAAGACG
*SEQ ID NO: 3: capturing 17-5p
CTACCTGCACTGTAAGCACTTTG
*SEQ ID NO: 4: capturing let7a-5p
AACTATACAACCTACTACCTCA

1. A method of analyzing a modification state of a microRNA, comprising:
(A) passing the microRNA between an electrode pair;
(B) detecting a tunneling current that is generated when the microRNA passes between the electrode pair; and
(C) analyzing the modification state based on the tunneling current.

2. The method of claim 1, wherein at least one of a modified position on a base sequence of the microRNA, a modified position on a chemical structure of the microRNA, a modification ratio of the microRNA, and a modification ratio at a specific modified position of the microRNA is identified.

3. The method of claim 1, wherein the modification comprises methylation.

4. The method of claim 1, wherein a base sequence of the microRNA is at least partially identified.

5. The method of claim 1, comprising referring to a result of analysis of the microRNA by a mass spectrometer to associate the modification state with the pattern of the tunneling current.

6.-10. (canceled)

11. A recording medium for storing program configured to implement, on a computer, a method of analyzing a microRNA, comprising: referring to accumulated data on a combination of a modification state of a microRNA and a pattern of a tunneling current obtained by tunneling current measurement on the microRNA to show a modification state of the microRNA of a subject from whom the microRNA has been obtained based on the pattern of a tunneling current obtained by tunneling current measurement on the microRNA.

12. (canceled)

13. A system for associating a modification state of a microRNA with a pattern of a tunneling current obtained by tunneling current measurement, comprising:
a mass spectrometer;
a tunneling current meter; and
an analysis/determination unit for analyzing and determining a modification state of a microRNA of interest by associating results of measuring the microRNA of interest by the mass spectrometer and the tunneling current measurement.

14.-15. (canceled)

16. The recording medium of claim 11, wherein the method further comprises: inputting a result of analysis of a microRNA by a mass spectrometer; and
determining a modification state of the microRNA by associating the result of analysis by mass spectrometry with the pattern of the tunneling current.

17. A method of claim 1, further comprising analyzing a condition of a subject based on the modification state, wherein the microRNA is derived from the subject.

18. The method of claim 17, wherein the condition of the subject from whom the microRNA has been obtained is analyzed by referring to accumulated data on a combination of the modification state and the pattern of the tunneling current to analyze a modification state of the microRNA based on the detected pattern of the tunneling current.

19. The method of claim 17, wherein a result of analyzing the condition of the subject from whom the microRNA has been obtained is shown in 15 minutes or less from the time the sample was subjected to tunneling current measurement.

20. The method of claim 17, wherein the condition of the subject includes cancer, an inflammatory bowel disease, Crohn's disease, diabetes, or a psychiatric disease.

21. The method of claim 17, wherein the condition of the subject includes the stage of cancer.

22. The method of claim 21, wherein the cancer is pancreatic cancer.

23. The method of claim 17, further comprising analyzing the base sequence based on the tunneling current and analyzing the condition of the subject based on the modification state and the base sequence.

24. The recording medium of claim 11, wherein the method further comprises analyzing a condition of a subject based on the modification state.

25. The recording medium of claim 24, wherein the condition of the subject includes cancer, an inflammatory bowel disease, Crohn's disease, diabetes, or a psychiatric disease.

26. The recording medium of claim 24, wherein the condition of the subject includes the stage of cancer.

27. The recording medium of claim 26, wherein the cancer is pancreatic cancer.

28. The recording medium of claim 24, wherein the method further comprises analyzing the base sequence based on the tunneling current and analyzing the condition of the subject based on the modification state and the base sequence.
